(19) United States
(12) Patent Application Publication
(10) Pub. No.: US 2012/0289703 A1
(43) Pub. Date: Nov. 15, 2012

(54) HALOGENATED PERYLENE-BASED SEMICONDUCTING MATERIALS

(75) Inventors: Helmut Reichelt, Neustadt (DE); Thomas Geßner, Heidelberg (DE); Chen Li, Mainz (DE); Klaus Müllen, Koln (DE); Glauco Battagliarin, Mainz (DE)

(73) Assignees: Max-Planck-Gesellschaft zur Foerd. der Wisse. e. V, Muenchen (DE); BASF SE, Ludwigshafen (DE)

(21) Appl. No.: 13/465,370

(22) Filed: May 7, 2012

Related U.S. Application Data
(60) Provisional application No. 61/484,687, filed on May 11, 2011.
(60) Provisional application No. 61/491,348, filed on May 31, 2011.

(51) Int. Cl. C07D 471/06

(52) U.S. Cl. 546/37

(57) ABSTRACT

The present invention provides a compound of formula

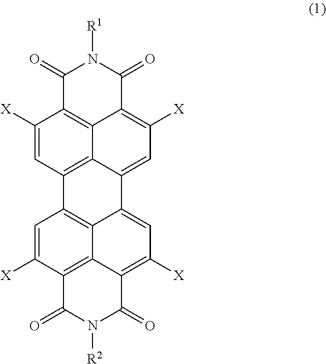

wherein X is —Cl, —Br or —I.

The compound of formula (1) is suitable for use as semiconducting material, in particular in electronic devices.

Organic semiconducting materials can be used in electronic devices such as organic photo-voltaic (OPV) cells, organic field-effect transistors (OFETs) and organic light emitting diodes (OLEDs).

For efficient and long lasting performance, it is desirable that the organic semiconducting material-based devices show high charge carrier mobility and high stability, in particular towards oxidation, under ambient conditions.

Furthermore, it is desirable that the organic semiconducting materials are compatible with liquid processing techniques as liquid processing techniques are convenient from the point of processability, and thus allow the production of low cost organic semiconducting material-based electronic devices. In addition, liquid processing techniques are also compatible with plastic substrates, and thus allow the production of light weight and flexible organic semiconducting material-based electronic devices.

Perylene bisimide-based organic semiconducting materials suitable for use in electronic devices are known in the art.

F. Würthner Chem. Commun. 2004, 1564-1579 describes perylene bisimide derivatives, for example

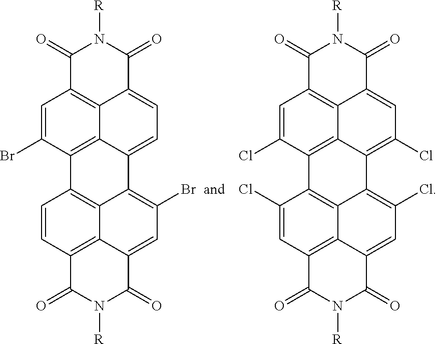

R. Schmidt, J. H. Oh, Y.-S. Sun, M. Deppisch, A.-M. Krause, K. Radacki, H. Braunschweig, M. Könemann, P. Erk, Z. Bao and F. Würthner J. Am. Chem. Soc. 2009, 131, 6215-6228 describes halogenated perylene bisimide derivatives, for example

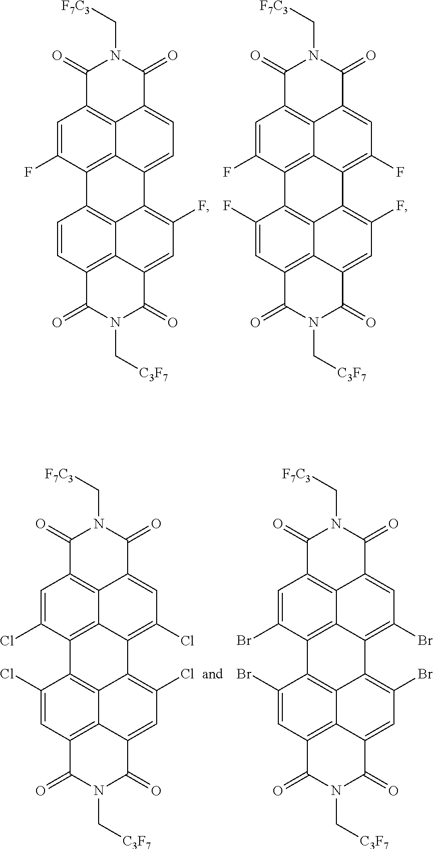

M. Gsänger, J. H. Oh, M. Könemann, H. W. Höffken, A.-M. Krause, Z. Bao, F. Würthner Angew. Chem. 2010, 122, 752-755 describes the following halogenated perylene bisimide

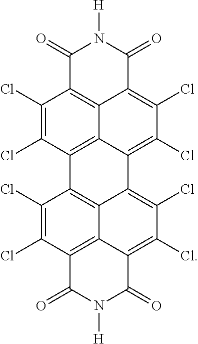

S. Nakazono, Y. Imazaki, H. Yoo, J. Yang, T. Sasamori, N. Tokitoh, T. Cédric, H. Kageyama, D. Kim, H. Shinokubo and A. Osuka Chem. Eur. J. 2009, 15, 7530-7533 describes the preparation of 2,5,8,11 tetraalkylated perylene tetracarboxylic acid bisimides from perylene tetracarboxylic acid bisimides

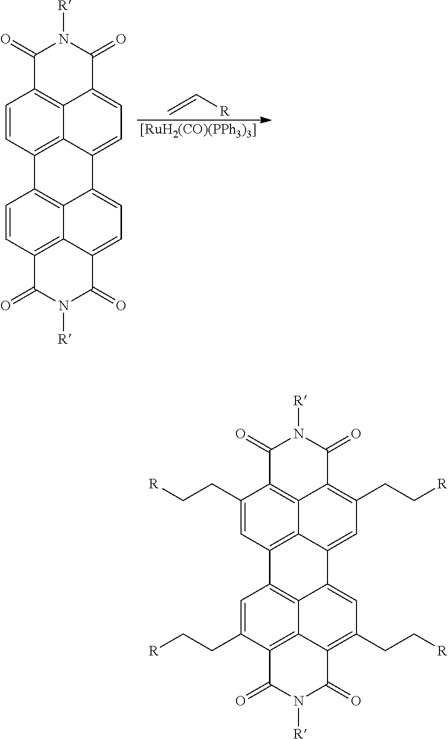

S. Nakanzono, S. Easwaramoorthi, D. Kim, H. Shinokubo, A. Osuka Org. Lett. 2009, 11, 5426 to 5429 describes the preparation of 2,5,8,11 tetraarylated perylene tetracarboxylic acid bisimides from perylene tetracarboxylic acid bisimides

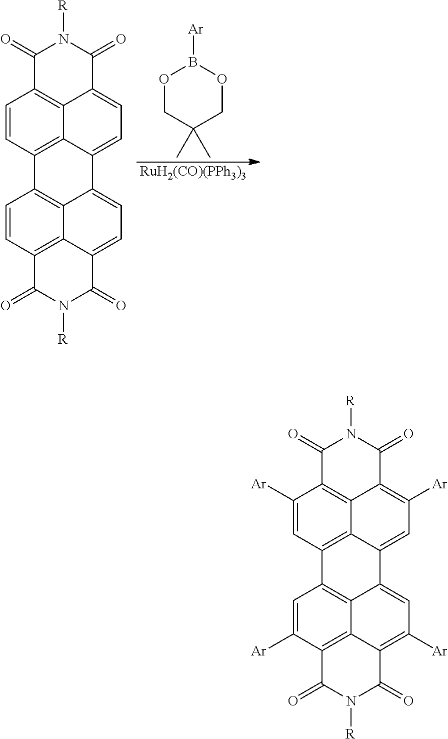

U.S. Pat. No. 7,282,275 B2 describes a composition that includes
a first compound of formula [EC—]ₙ—Ar¹ (I), wherein
   Ar¹ is a first aromatic core and is a divalent, trivalent or tetravalent radical of a long list of formulae, including

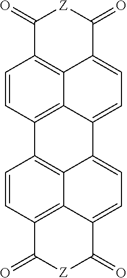

that is unsubstituted or substituted with a long list of substituents, including fluoro,
EC is a first end capping group and is a monovalent radical of a long list of formulae,
n is an integer of 2 to 4
Z is NH or CH₂, and
a second compound having an aromatic radical that comprises the first aromatic core of the first compound, a second end capping group that comprises the first end capping group of the first compound, a divalent radical that comprises a divalent radical of the first end capping group, or a combination thereof,

wherein the composition is amorphous and solution proccessible.

U.S. Pat. No. 7,355,198 B2 describes an organic thin film transistor (OFET), which interposes an organic acceptor film between source and drain electrodes and an organic semiconductor film. The organic semiconductor film is formed of pentacene. In particular, the organic acceptor film is formed of at least one electron withdrawing material selected from a long list of compounds, including N,N′-bis(di-tert-butyphenyl)-3,4,9,10-perylenedicarboximide.

U.S. Pat. No. 7,326,956 B2 describes a thin film transitor comprising a layer of organic semiconductor material comprising tetracarboxylic diimide perylene-based compound having attached to each of the imide nitrogen atoms a carbocyclic or heterocyclic aromatic ring system substituted with one or more fluorine containing groups. In one embodiment the fluorine-containing N,N′-diaryl perylene-based tetracarboxylic diimide compound is represented by the following structure:

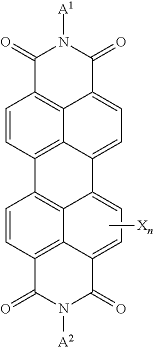

wherein A¹ and A² are independently carbocyclic and/or heterocyclic aromatic ring systems comprising at least one aromatic ring in which one or more hydrogen atoms are substituted with at least one fluorine-containing group. The perylene nucleus can be optionally substituted with up to eight independently selected X groups, wherein n is an integer from 0 to 8. The X substituent groups on the perylene can include a long list of substituents, including halogens such as fluorine or chlorine.

U.S. Pat. No. 7,671,202 B2 describes n-type semiconductor compounds of formula

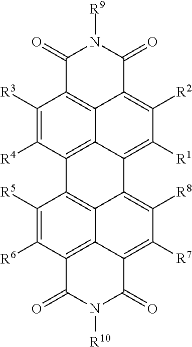

wherein each R¹ to R⁸ can be independently selected from H, an electron-withdrawing substituent and a moiety comprising such substituent. Electron-withdrawing substitutents include a long list of substituents, including cyano. R⁹ and R¹⁰ are independently selected from H, alkyl, substituted alkyl, cycloalkyl, substituted cycloalkyl, aryl, substituted aryl, polycyclic aryl and/or substituted polycyclic aryl moieties.

WO 2005/124453 describes perylenetetracarboxylic diimide charge-transfer materials, for example a perylenetetracarboxylic diimide charge-transfer material having formula

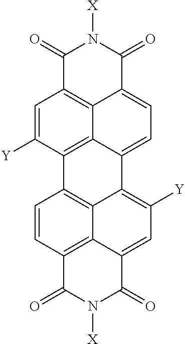

wherein Y in each instance can be independently selected from H, CN, acceptors, donors and a polymerizable group; and X in each instance can be independently selected from a large group of listed compounds.

WO 2008/063609 describes a compound having the following formula

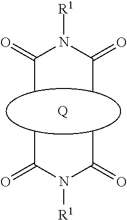

wherein Q can be

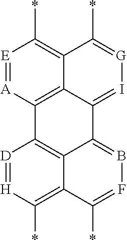

wherein A, B, I, D, E, F, G and H are independently selected from a group of substituents, including, CH and CR^{a}, wherein R^{a} can be selected from a list of substituents, including halogen. For example, A, B, I, D, E, F, G and H can be independently CH, C—Br or C—CN.

WO 2009/098252 describes semiconducting compounds having formula

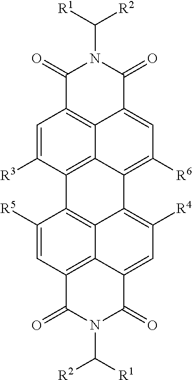

wherein R¹ and R² at each occurrence independently are selected from a large list of groups, including H, C₁₋₃₀-alkyl and C₂₋₃₀-alkenyl; and R³, R⁴, R⁵ and R⁶ are independently H or an electron-withdrawing group. In certain embodiments, R³, R⁴, R⁵ and R⁶ can be independently from each other H, F, Cl, Br, I or CN.

WO 2009/144205 describes bispolycyclic rylene-based semiconducting compounds, which can be prepared from a compound of formula

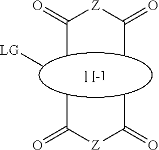

wherein LG is a leaving group, including Cl, Br or I,

π-1 can be

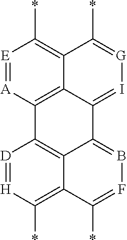

wherein A, B, I, D, E, F, G and H are independently selected from a group of substituents, including, CH and CR^{a}, wherein R^{a} can be selected from a list of substituents, including halogen.

So far, it has not been possible to prepare 2,5,8,11-tetrahalogenoperylene-bis(dicarboximides).

It was the object of the present invention to provide new perylene-based semiconducting materials.

The object is solved by the compound of claim 1, the process of claim 5, and the electronic device of claim 6.

The perylene-based semiconducting compound of the present invention is of formula

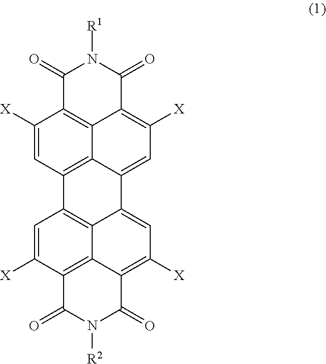

wherein
R¹ and R² are independently from each other selected from the group consisting of H, C₁₋₃₀-alkyl optionally substituted with 1 to 30 substituents R^{a}, C₂₋₃₀-alkenyl optionally substituted with 1 to 30 substituents R^{a}, C₂₋₃₀-alkynyl optionally substituted with 1 to 30 substituents R^{a}, C₃₋₁₀-cycloalkyl optionally substituted with 1 to 10 substituents R^{b}, C₅₋₁₀-cycloalkenyl optionally substituted with 1 to 10 substituents R^{b}, 3-14 membered cycloheteroalkyl optionally substituted with 1 to 8 substituents R^{b}, C₆₋₁₄-aryl optionally substituted with 1 to 8 substituents R^{c} and 5-14 membered heteroaryl optionally substituted with 1 to 8 substituents R^{c},
   wherein
   R^{a} at each occurrence are independently from each other selected from the group consisting of halogen, —CN, —NO₂, —N₃, —OH, C₁₋₃₀-alkoxy optionally substituted with 1 to 6 substituents Rⁱ, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl optionally substituted with 1 to 30 substituents Rⁱ, —SO₂—C₁₋₃₀-alkyl optionally substituted with 1 to 30 substituents Rⁱ, —NH₂, —NHR³, —NR³R⁴, —[NR³R⁴R⁵]⁺, —NH—COR³, —COON, —COOR³, —CONH₂, —CONHR³, —CONR³R⁴, —CO—H, —COR³, C₃₋₁₀-cycloalkyl optionally substituted with 1 to 10 substituents Rⁱⁱ, C₅₋₁₀-cycloalkenyl optionally substituted with 1 to 10 substituents Rⁱⁱ, 3-14 membered cycloheteroalkyl optionally substituted with 1 to 10 substituents Rⁱⁱ, C₆₋₁₄-aryl optionally substituted with 1 to 8 substituents Rⁱⁱⁱ and 5-14 membered heteroaryl optionally substituted with 1 to 8 substituents Rⁱⁱⁱ;
   R^{b} at each occurrence are independently from each other selected from the group consisting of halogen, —CN, —NO₂, —OH, C₁₋₃₀-alkoxy optionally substituted with 1 to 30 substituents Rⁱ, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl optionally substituted with 1 to 30 substituents Rⁱ, —NH₂, —NHR³, —NR³R⁴, —[NR³R⁴R⁵]⁺, —NH—COR³, —COOH, —COOR³, —CONH₂, —CONHR³, —CONR³R⁴, —CO—H, —COR³, C₁₋₃₀-alkyl optionally substituted with 1 to 30 substituents Rⁱ, C₂₋₃₀-alkenyl optionally substituted with 1 to 30 substituents Rⁱ, C₂₋₃₀-alkynyl optionally substituted with 1 to 30 substituents Rⁱ, C₃₋₁₀-cycloalkyl optionally substituted with 1 to 10 substituents Rⁱⁱ, C₅₋₁₀-cycloalkenyl optionally substituted with 1 to 10 substituents Rⁱⁱ, 3-14 membered cycloheteroalkyl optionally substituted with 1 to 10 substituents Rⁱⁱ, C₆₋₁₄-aryl optionally substituted with 1 to 8 substituents Rⁱⁱⁱ and 5-14 membered heteroaryl optionally substituted with 1 to 8 substituents Rⁱⁱⁱ;
   R^{c} at each occurrence are independently from each other selected from the group consisting of halogen, —CN, —NO₂, —N₃, —OH, C₁₋₃₀-alkoxy optionally substituted with 1 to 30 substituents Rⁱ, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl optionally substituted with 1 to 30 substituents Rⁱ, —SO₂—C₁₋₃₀-alkyl optionally substituted with 1 to 30 substituents Rⁱ, —NH₂, —NHR³, —NR³R⁴, —[NR³R⁴R⁵]⁺, —NH—COR³, —COOH, —COOR³, —CONH₂, —CONHR³, —CONR³R⁴, —CO—H, —COR³, C₁₋₃₀-alkyl optionally substituted with 1 to 30 substituents Rⁱ, C₂₋₃₀-alkenyl optionally substituted with 1 to 30 substituents Rⁱ, C₂₋₃₀-alkynyl optionally substituted with 1 to 30 substituents Rⁱ, C₃₋₁₀-cycloalkyl optionally substituted with 1 to 10 substituents Rⁱⁱ, C₅₋₁₀-cycloalkenyl optionally substituted with 1 to 10 substituents Rⁱⁱ, 3-14 membered cycloheteroalkyl optionally substituted with 1 to 10 substituents Rⁱⁱ, C₆₋₁₄-aryl optionally substituted with 1 to 8 substituents Rⁱⁱⁱ and 5-14 membered heteroaryl optionally substituted with 1 to 8 substituents Rⁱⁱⁱ;
      wherein
      R³, R⁴ and R⁵ at each occurrence are independently from each other selected from the group consisting of C₁₋₃₀-alkyl optionally substituted with 1 to 30 substituents Rⁱ, C₂₋₃₀-alkenyl optionally substituted with 1 to 30 substituents Rⁱ, C₂₋₃₀-alkynyl optionally substituted with 1 to 30 substituents Rⁱ, C₃₋₁₀-cycloalkyl optionally substituted with 1 to 10 substituents Rⁱⁱ, C₅₋₁₀-cycloalkenyl optionally substituted with 1 to 10 substituents Rⁱⁱ, 3-14 membered cycloheteroalkyl optionally substituted with 1 to 10 substituents Rⁱⁱ, C₆₋₁₄-aryl optionally substituted with 1 to 8 substituents Rⁱⁱⁱ and 5-14 membered heteroaryl optionally substituted with 1 to 8 substituents Rⁱⁱⁱ,
      Rⁱ at each occurrence are independently from each other selected from the group consisting of halogen, —CN, —NO₂, —N₃, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl, —SO₂—C₁₋₃₀-alkyl, —NH₂, —NHR⁶, —NR⁶R⁷, —[NR⁶R⁷R⁸]⁺, —NH—COR⁶, —COOH, —COOR⁶, —CONH₂, —CONHR⁶, —CONR⁶R⁷, —CO—H, —COR⁶, C₃₋₁₀-cycloalkyl, C₅₋₁₀-cycloalkenyl, 3-14 membered cycloheteroalkyl, C₆₋₁₄-aryl and 5-14 membered heteroaryl,
      Rⁱⁱ at each occurrence are independently from each other selected from the group consisting of halogen, —CN, —NO₂, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR⁶, —S—C₁₋₃₀-alkyl, —NH₂, —NHR⁶, —NR⁶R⁷, —[NR⁶R⁷R⁸]⁺, —NH—COR⁶, —COOH, —COOR⁶, —CONH₂, —CONHR⁶, —CONR⁶R⁷, —CO—H, —COR⁶, C₁₋₃₀-alkyl, C₂₋₃₀-alkenyl, C₂₋₃₀-alkynyl, C₃₋₁₀-cycloalkyl, C₅₋₁₀-cycloalkenyl, 3-14 membered cycloheteroalkyl, C₆₋₁₄-aryl and 5-14 membered heteroaryl,
      Rⁱⁱⁱ at each occurrence are independently from each other selected from the group consisting of halogen, —CN, —NO₂, —N₃, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR⁶, —S—C₁₋₃₀-alkyl, —SO₂—C₁₋₃₀-alkyl, —NH₂, —NHR⁶, —NR⁶R⁷, —[NR⁶R⁷R⁸]⁺, —NH—COR⁶, —COOH, —COOR⁶, —CONH₂, —CONHR⁶, —CONR⁶R⁷, —CO—H, —COR⁶, C₁₋₃₀-alkyl, C₂₋₃₀-alkenyl, C₂₋₃₀-alkynyl, C₃₋₁₀-cycloalkyl, C₅₋₁₀-cycloalkenyl, 3-14 membered cycloheteroalkyl, C₆₋₁₄-aryl and 5-14 membered heteroaryl,
         wherein
         R⁶, R⁷ and R⁸ at each occurrence are independently from each other selected from the group consisting of C₁₋₃₀-alkyl, C₂₋₃₀-alkenyl, C₂₋₃₀-alkynyl, C₃₋₁₀-cycloalkyl, C₅₋₁₀-cycloalkenyl, 3-14 membered cycloheteroalkyl, C₆₋₁₄-aryl and 5-14 membered heteroaryl,

and

X is —Cl, —Br or —I.

C₁₋₁₀-alkyl and C₁₋₃₀-alkyl can be branched or unbranched. Examples of C₁₋₁₀-alkyl are methyl, ethyl, n-propyl, isopropyl, n-butyl, sec-butyl, isobutyl, tert-butyl, n-pentyl, neopentyl, isopentyl, n-(1-ethyl)propyl, n-hexyl, n-heptyl, n-octyl, n-(2-ethyl)hexyl, n-nonyl and n-decyl. Examples of C₃₋₈-alkyl are n-propyl, isopropyl, n-butyl, sec-butyl, isobutyl, tert-butyl, n-pentyl, neopentyl, iso-pentyl, n-(1-ethyl)propyl, n-hexyl, n-heptyl, n-octyl and n-(2-ethyl)hexyl. Examples of C₁₋₃₀-alkyl are C₁₋₁₀-alkyl, and n-undecyl, n-dodecyl, n-tridecyl, n-tetradecyl, n-pentadecyl, n-hexadecyl, n-heptadecyl, n-octadecyl, n-nonadecyl and n-icosyl (C₂₀), n-docosyl (C₂₂), n-tetracosyl (C₂₄), n-hexacosyl (C₂₆), n-octacosyl (C₂₈) and n-triacontyl (C₃₀). Examples of C₃₋₂₅-alkyl branched at the C attached to the N of formula I are isopropyl, sec-butyl, n-(1-methyl)propyl, n-(1-ethyl)propyl, n-(1-methyl)butyl, n-(1-ethyl)butyl, n-(1-propyl)butyl, n-(1-methyl)pentyl, n-(1-ethyl)pentyl, n-(1-propyl)pentyl, n-(1-butyl)pentyl, n-(1-butyl)hexyl, n-(1-pentyl)hexyl, n-(1-hexyl)heptyl, n-(1-heptyl)octyl, n-(1-octyl)nonyl, n-(1-nonyl)decyl, n-(1-decyl)undecyl, n-(1-undecyl)dodecyl and n-(1-dodecyl)tridecyl.

C₂₋₃₀-alkenyl can be branched or unbranched. Examples of C₂₋₃₀-alkenyl are vinyl, propenyl, cis-2-butenyl, trans-2-butenyl, 3-butenyl, cis-2-pentenyl, trans-2-pentenyl, cis-3-pentenyl, trans-3-pentenyl, 4-pentenyl, 2-methyl-3-butenyl, hexenyl, heptenyl, octenyl, nonenyl and docenyl, linoleyl (C₁₈), linolenyl (C₁₈), oleyl (C₁₈), arachidonyl (C₂₀), and erucyl (C₂₂).

C₂₋₃₀-alkynyl can be branched or unbranched. Examples of C₂₋₃₀-alkynyl are ethynyl, 2-propynyl, 2-butynyl, 3-butynyl, pentynyl, hexynyl, heptynyl, octynyl, nonynyl and decynyl, undecynyl, dodecynyl, undecynyl, dodecynyl, tridecynyl, tetradecynyl, pentadecynyl, hexadecynyl, heptadecynyl, octadecynyl, nonadecynyl and icosynyl (C₂₀).

Examples of C₃₋₁₀-cycloalkyl are preferably monocyclic C₃₋₁₀-cycloalkyls such as cyclopropyl, cyclobutyl, cyclopentyl, cyclohexyl, cycloheptyl and cyclooctyl, but include also polycyclic C₃₋₁₀-cycloalkyls such as decalinyl, norbornyl and adamantyl.

Examples of C₅₋₁₀-cycloalkenyl are preferably monocyclic C₅₋₁₀-cycloalkenyls such as cyclopentenyl, cyclohexenyl, cyclohexadienyl and cycloheptatrienyl, but include also polycyclic C₅₋₁₀-cycloalkenyls.

Examples of 3-14 membered cycloheteroalkyl are monocyclic 3-8 membered cycloheteroalkyl and polycyclic, for example bicyclic 7-12 membered cycloheteroalkyl.

Examples of monocyclic 3-8 membered cycloheteroalkyl are monocyclic 5 membered cycloheteroalkyl containing one heteroatom such as pyrrolidinyl, 1-pyrrolinyl, 2-pyrrolinyl, 3-pyrrolinyl, tetrahydrofuryl, 2,3-dihydrofuryl, tetrahydrothiophenyl and 2,3-dihydrothiophenyl, monocyclic 5 membered cycloheteroalkyl containing two heteroatoms such as imidazolidinyl, imidazolinyl, pyrazolidinyl, pyrazolinyl, oxazolidinyl, oxazolinyl, isoxazolidinyl, isoxazolinyl, thiazolidinyl, thiazolinyl, isothiazolidinyl and isothiazolinyl, monocyclic 5 membered cycloheteroalkyl containing three heteroatoms such as 1,2,3-triazolyl, 1,2,4-triazolyl and 1,4,2-dithiazolyl, monocyclic 6 membered cycloheteroalkyl containing one heteroatom such as piperidyl, piperidino, tetrahydropyranyl, pyranyl, thianyl and thiopyranyl, monocyclic 6 membered cycloheteroalkyl containing two heteroatoms such as piperazinyl, morpholinyl and morpholino and thiazinyl, monocyclic 7 membered cycloheteroalkyl containing one hereoatom such as azepanyl, azepinyl, oxepanyl, thiepanyl, thiapanyl, thiepinyl, and monocyclic 7 membered cycloheteroalkyl containing two hereoatom such as 1,2-diazepinyl and 1,3-thiazepinyl.

An example of a bicyclic 7-12 membered cycloheteroalkyl is decahydronaphthyl.

C₆₋₁₄-aryl can be monocyclic or polycyclic. Examples of C₆₋₁₄-aryl are monocyclic C₆-aryl such as phenyl, bicyclic C₉₋₁₀-aryl such as 1-naphthyl, 2-naphthyl, indenyl, indanyl and tetrahydronaphthyl, and tricyclic C₁₂₋₁₄-aryl such as anthryl, phenanthryl, fluorenyl and s-indacenyl.

5-14 membered heteroaryl can be monocyclic 5-8 membered heteroaryl, or polycyclic 7-14 membered heteroaryl, for example bicyclic 7-12 membered or tricyclic 9-14 membered heteroaryl.

Examples of monocyclic 5-8 membered heteroaryl are monocyclic 5 membered heteroaryl containing one heteroatom such as pyrrolyl, furyl and thiophenyl, monocyclic 5 membered heteroaryl containing two heteroatoms such as imidazolyl, pyrazolyl, oxazolyl, isoxazolyl, thiazolyl, isothiazolyl, monocyclic 5 membered heteroaryl containing three heteroatoms such as 1,2,3-triazolyl, 1,2,4-triazolyl and oxadiazolyl, monocyclic 5 membered heteroaryl containing four heteroatoms such as tetrazolyl, monocyclic 6 membered heteroaryl containing one heteroatom such as pyridyl, monocyclic 6 membered heteroaryl containing two heteroatoms such as pyrazinyl, pyrimidinyl and pyridazinyl, monocyclic 6 membered heteroaryl containing three heteroatoms such as 1,2,3-triazinyl, 1,2,4-triazinyl and 1,3,5-triazinyl, monocyclic 7 membered heteroaryl containing one heteroatom such as azepinyl, and monocyclic 7 membered heteroaryl containing two heteroatoms such as 1,2-diazepinyl.

Examples of bicyclic 7-12 membered heteroaryl are bicyclic 9 membered heteroaryl containing one heteroatom such as indolyl, isoindolyl, indolizinyl, indolinyl, benzofuryl, isobenzofuryl, benzothiophenyl and isobenzothiophenyl, bicyclic 9 membered heteroaryl containing two heteroatoms such as indazolyl, benzimidazolyl, benzimidazolinyl, benzoxazolyl, benzisooxazolyl, benzthiazolyl, benzisothiazolyl, furopyridyl and thienopyridyl, bicyclic 9 membered heteroaryl containing three heteroatoms such as benzotriazolyl, benzoxadiazolyl, oxazolopyridyl, isooxazolopyridyl, thiazolopyridyl, isothiazolopyridyl and imidazopyridyl, bicyclic 9 membered heteroaryl containing four heteroatoms such as purinyl, bicyclic 10 membered heteroaryl containing one heteroatom such as quinolyl, isoquinolyl, chromenyl and chromanyl, bicyclic 10 membered heteroaryl containing two heteroatoms such as quinoxalinyl, quinazolinyl, cinnolinyl, phthalazinyl, 1,5-naphthyridinyl and 1,8-naphthyridinyl, bicyclic 10 membered heteroaryl containing three heteroatoms such as pyridopyrazinyl, pyridopyrimidinyl and pyridopyridazinyl, and bicyclic 10 membered heteroaryl containing four heteroatoms such as pteridinyl.

Examples of tricyclic 9-14 membered heteroaryls are dibenzofuryl, acridinyl, phenoxazinyl, 7H-cyclopenta[1,2-b:3,4-b′]dithiophenyl and 4H-cyclopenta[2,1-b:3,4-b′]dithiophenyl.

Examples of halogen are —F, —Cl, —Br and —I.

Examples of C₁₋₃₀-alkoxy are methoxy, ethoxy, n-propoxy, isopropoxy, n-butoxy, sec-butoxy, isobutoxy, tert-butoxy, n-pentoxy, neopentoxy, isopentoxy, hexoxy, n-heptoxy, n-octoxy, n-nonoxy, n-decoxy, n-undecoxy, n-dodecoxy, n-tridecoxy, n-tetradecoxy, n-pentadecoxy, n-hexadecoxy, n-heptadecoxy, n-octadecoxy and n-nonadecoxy.

Examples of C₂₋₅-alkylene are ethylene, propylene, butylene and pentylene.

Preferably,
R¹ and R² are independently from each other selected from the group consisting of H, C₁₋₃₀-alkyl optionally substituted with 1 to 30 substituents R^{a}, C₂₋₃₀-alkenyl optionally substituted with 1 to 30 substituents R^{a}, C₃₋₁₀-cycloalkyl optionally substituted with 1 to 10 substituents R^{b}, and C₆₋₁₄-aryl optionally substituted with 1 to 8 substituents R^{c},
   wherein
   R^{a} at each occurrence are independently from each other selected from the group consisting of halogen, —CN, —NO₂, —N₃, —OH, C₁₋₃₀-alkoxy optionally substituted with 1 to 6 substituents Rⁱ, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl optionally substituted with 1 to 30 substituents Rⁱ, —SO₂—C₁₋₃₀-alkyl optionally substituted with 1 to 30 substituents Rⁱ, —NH₂, —NHR³, —NR³R⁴, —[NR³R⁴R⁵]⁺, —NH—COR³, —COOH, —COOR³, —CONH₂, —CONHR³, —CONR³R⁴, —CO—H, —COR³, C₃₋₁₀-cycloalkyl optionally substituted with 1 to 10 substituents Rⁱⁱ, and C₆₋₁₄-aryl optionally substituted with 1 to 8 substituents Rⁱⁱⁱ;
   R^{b} at each occurrence are independently from each other selected from the group consisting of halogen, —CN, —NO₂, —OH, C₁₋₃₀-alkoxy optionally substituted with 1 to 30 substituents Rⁱ, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl optionally substituted with 1 to 30 substituents Rⁱ, —NH₂, —NHR³, —NR³R⁴, —[NR³R⁴R⁵]⁺, —NH—COR³, —COOH, —COOR³, —CONH₂, —CONHR³, —CONR³R⁴, —CO—H, —COR³, C₁₋₃₀-alkyl optionally substituted with 1 to 30 substituents Rⁱ, C₂₋₃₀-alkenyl optionally substituted with 1 to 30 substituents Rⁱ, C₃₋₁₀-cycloalkyl optionally substituted with 1 to 10 substituents Rⁱⁱ, and C₆₋₁₄-aryl optionally substituted with 1 to 8 substituents Rⁱⁱⁱ;
   R^{c} at each occurrence are independently from each other selected from the group consisting of halogen, —CN, —NO₂, —N₃, —OH, C₁₋₃₀-alkoxy optionally substituted with 1 to 30 substituents Rⁱ, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl optionally substituted with 1 to 30 substituents Rⁱ, —SO₂—C₁₋₃₀-alkyl optionally substituted with 1 to 30 substituents Rⁱ, —NH₂, —NHR³, —NR³R⁴, —[NR³R⁴R⁵]⁺, —NH—COR³, —COOH, —COOR³, —CONH₂, —CONHR³, —CONR³R⁴, —CO—H, —COR³, C₁₋₃₀-alkyl optionally substituted with 1 to 30 substituents Rⁱ, C₂₋₃₀-alkenyl optionally substituted with 1 to 30 substituents Rⁱ, C₃₋₁₀-cycloalkyl optionally substituted with 1 to 10 substituents Rⁱⁱ, and C₆₋₁₄-aryl optionally substituted with 1 to 8 substituents Rⁱⁱⁱ;
      wherein
      R³, R⁴ and R⁵ at each occurrence are independently from each other selected from the group consisting of C₁₋₃₀-alkyl optionally substituted with 1 to 30 substituents Rⁱ,
      C₂₋₃₀-alkenyl optionally substituted with 1 to 30 substituents Rⁱ, C₃₋₁₀-cycloalkyl optionally substituted with 1 to 10 substituents Rⁱⁱ, and C₆₋₁₄-aryl optionally substituted with 1 to 8 substituents Rⁱⁱⁱ,
      Rⁱ at each occurrence are independently from each other selected from the group consisting of halogen, —CN, —NO₂, —N₃, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl, —SO₂—C₁₋₃₀-alkyl, —NH₂, —NHR⁶, —NR⁶R⁷, —[NR⁶R⁷R⁸]⁺, —NH—COR⁶, —COOH, —COOR⁶, —CONH₂, —CONHR⁶, —CONR⁶R⁷, —CO—H, —COR⁶, C₃₋₁₀-cycloalkyl, and C₆₋₁₄-aryl,
      Rⁱⁱ at each occurrence are independently from each other selected from the group consisting of halogen, —CN, —NO₂, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR⁶, —S—C₁₋₃₀-alkyl, —NH₂, —NHR⁶, —NR⁶R⁷, —[NR⁶R⁷R⁸]⁺, —NH—COR⁶, —COOH, —COOR⁶, —CONH₂, —CONHR⁶, —CONR⁶R⁷, —CO—H, —COR⁶, C₁₋₃₀-alkyl, C₂₋₃₀-alkenyl, C₃₋₁₀-cycloalkyl, and C₆₋₁₄-aryl,
      Rⁱⁱⁱ at each occurrence are independently from each other selected from the group consisting of halogen, —CN, —NO₂, —N₃, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR⁶, —S—C₁₋₃₀-alkyl, —SO₂—C₁₋₃₀-alkyl, —NH₂, —NHR⁶, —NR⁶R⁷, —[NR⁶R⁷R⁸]⁺, —NH—COR⁶, —COOH, —COOR⁶, —CONH₂, —CONHR⁶, —CONR⁶R⁷, —CO—H, —COR⁶, C₁₋₃₀-alkyl, C₂₋₃₀-alkenyl, C₃₋₁₀-cycloalkyl, and C₆₋₁₄-aryl,
         wherein
         R⁶, R⁷ and R⁸ at each occurrence are independently from each other selected from the group consisting of C₁₋₃₀-alkyl, C₂₋₃₀-alkenyl, C₃₋₁₀-cycloalkyl, and C₆₋₁₄-aryl,

and

X is —Cl, —Br or I.

More preferably,
R¹ and R² are independently from each other C₁₋₃₀-alkyl optionally substituted with 1 to 30 substituents R^{a},
   wherein
   R^{a} at each occurrence are independently from each other selected from the group consisting of halogen, —CN, —NO₂, —N₃, —OH, C₁₋₃₀-alkoxy optionally substituted with 1 to 6 substituents Rⁱ, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl optionally substituted with 1 to 30 substituents Rⁱ, —SO₂—C₁₋₃₀-alkyl optionally substituted with 1 to 30 substituents Rⁱ, —NH₂, —NHR³, —NR³R⁴, —[NR³R⁴R⁵]⁺, —NH—COR³, —COOH, —COOR³, —CONH₂, —CONHR³, —CONR³R⁴, —CO—H, —COR³, C₃₋₁₀-cycloalkyl optionally substituted with 1 to 10 substituents Rⁱⁱ, and C₆₋₁₄-aryl optionally substituted with 1 to 8 substituents Rⁱⁱⁱ;
      wherein
      R³, R⁴ and R⁵ at each occurrence are independently from each other selected from the group consisting of C₁₋₃₀-alkyl optionally substituted with 1 to 30 substituents Rⁱ, C₂₋₃₀-alkenyl optionally substituted with 1 to 30 substituents Rⁱ, C₃₋₁₀-cycloalkyl optionally substituted with 1 to 10 substituents Rⁱⁱ, and C₆₋₁₄-aryl optionally substituted with 1 to 8 substituents Rⁱⁱⁱ,
      Rⁱ at each occurrence are independently from each other selected from the group consisting of halogen, —CN, —NO₂, —N₃, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl, —NH₂, —NHR⁶, —SO₂—C₁₋₃₀-alkyl, —NR⁶R⁷, —[NR⁶R⁷R⁸]⁺, —NH—COR⁶, —COON, —COOR⁶, —CONH₂, —CONHR⁶, —CONR⁶R⁷, —CO—H, —COR⁶, C₃₋₁₀-cycloalkyl, and C₆₋₁₄-aryl,
      Rⁱⁱ at each occurrence are independently from each other selected from the group consisting of halogen, —CN, —NO₂, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR⁶, —S—C₁₋₃₀-alkyl, —NH₂, —NHR⁶, —NR⁶R⁷, —[NR⁶R⁷R⁸]⁺, —NH—COR⁶, —COOH, —COOR⁶, —CONH₂, —CONHR⁶, —CONR⁶R⁷, —CO—H, —COR⁶, C₁₋₃₀-alkyl, C₂₋₃₀-alkenyl, C₃₋₁₀-cycloalkyl, and C₆₋₁₄-aryl,
      Rⁱⁱⁱ at each occurrence are independently from each other selected from the group consisting of halogen, —CN, —NO₂, —N₃, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR⁶, —S—C₁₋₃₀-alkyl, —SO₂—C₁₋₃₀-alkyl, —NH₂, —NHR⁶, —NR⁶R⁷, —[NR⁶R⁷R⁸]⁺, —NH—COR⁶, —COON, —COOR⁶, —CONH₂, —CONHR⁶, —CONR⁶R⁷, —CO—H, —COR⁶, C₁₋₃₀-alkyl, C₂₋₃₀-alkenyl, C₃₋₁₀-cycloalkyl, and C₆₋₁₄-aryl,
         wherein
         R⁶, R⁷ and R⁸ at each occurrence are independently from each other selected from the group consisting of C₁₋₃₀-alkyl, C₂₋₃₀-alkenyl, C₃₋₁₀-cycloalkyl, and C₆₋₁₄-aryl,

and

X is —Cl, —Br or —I.

Most preferably,

R¹ and R² are independently from each other C₃₋₂₅-alkyl branched at the C attached to the N of formula 1

and

X is —Cl, —Br or —I.

Particular preferred are the compounds of formulae

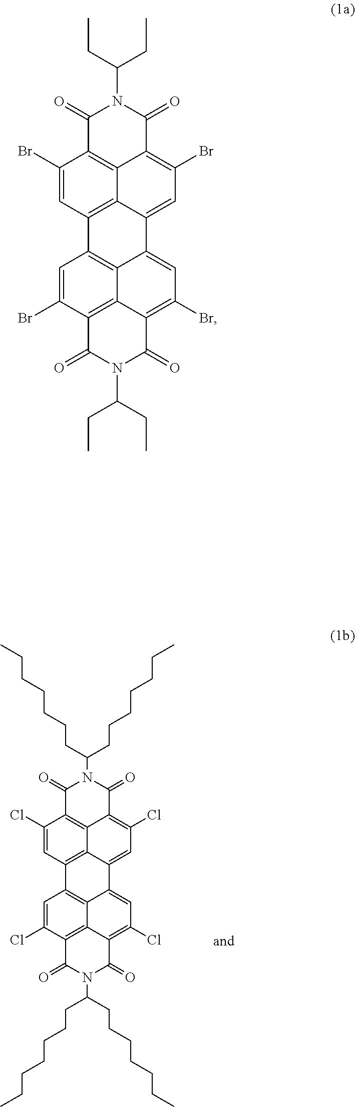

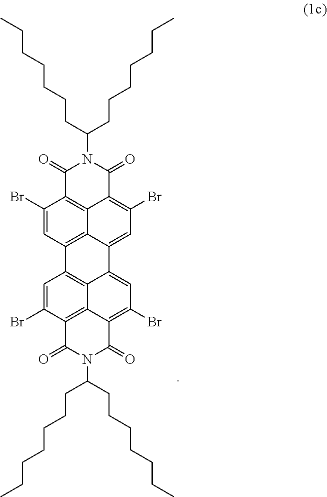

Also part of the invention, is a process for the preparation of the compound of formula

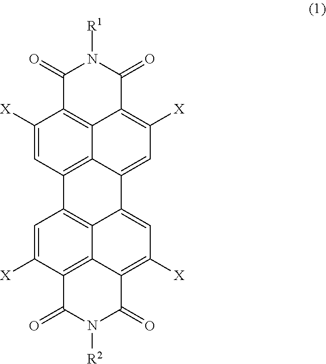

wherein R¹ and R² are as defined above,

which process comprises the steps of

(i) treating a compound of formula (2) with a boron-containing compound of formula (3) in the presence of a transition metal-containing catalyst to form a boron-containing compound of formula (4)

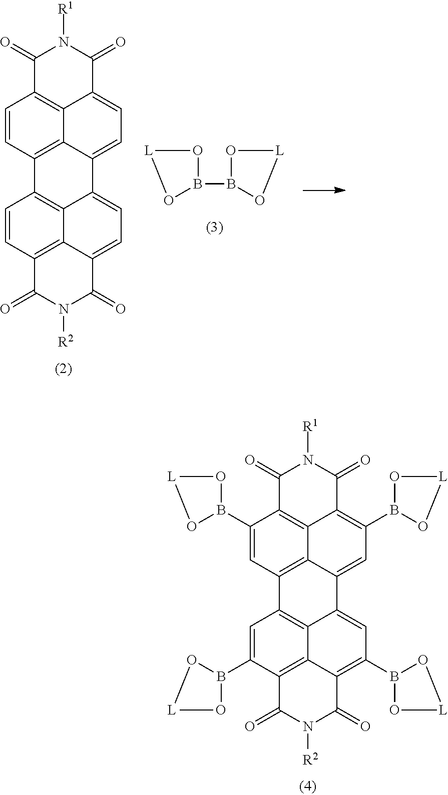

wherein R¹ and R² are as defined above, and L is a linking group,

and

(ii) treating the boron-containing compound of formula (4) with a Cl-, Br- or I-source in order to form the compound of formula (1).

L is preferably C₂₋₅-alkylene, which can be optionally substituted with 1 to 6 C₁₋₁₀-alkyl groups. More preferably L is ethylene or propylene and is substituted with 2 to 4 methyl groups.

The transition metal-containing catalyst can be an iridium-containing catalyst such as [Ir(cod)OMe]₂, or, preferably, a ruthenium-containing catalyst, such as RuH₂(CO)(PPh₃)₃.

If the transition metal-containing catalyst is an iridium-containing catalyst, the first step can be performed in the presence of a base such as di-tert-butylbipyridine. If the transition metal-containing catalyst is an iridium-containing catalyst, the first step is usually performed in a suitable organic solvent such as tetrahydrofuran or 1,4-dioxane. If the transition metal-containing catalyst is an iridium-containing catalyst, the first step is usually performed at elevated temperatures, such as at temperatures from 60 to 110° C. In principal, if the transition metal-containing catalyst is an iridium-containing catalyst, the first step can be performed in analogy to the method described by C. W. Liskey; X. Liao; J. F. Hartwig in J. Am. Chem. Soc. 2010, 132, 11389-11391, and by I. A. I. Mkhalid, J. H. Barnard, T. B. Marder, J. M. Murphy and J. F. Hartwig in Chem. Rev. 2010, 110, 890-931.

If the transition metal-containing catalyst is a ruthenium-containing catalyst, the first step is usually performed in a suitable organic solvent such as toluene, pinacolone and mesitylene or mixtures thereof. If the transition metal-containing catalyst is ruthenium-containing catalyst, the first step is usually performed at elevated temperatures, such as at temperatures from 120 to 160° C.

The Cl-source source can be Cu(II)Cl₂. The Br-source source can be Cu(II)Br₂. The I-source source can be NaI in combination with chloroamine T.

The second step is usually performed in a suitable solvent such as water, methanol, THF and dioxane, or mixtures thereof. The second step is usually performed at elevated temperatures, such as at temperatures from 40 to 140° C. When Cu(II)Cl₂ and Cu(II)Br₂ are used, the second step is preferably performed at elevated temperatures, such as at temperatures from 80 to 140° C. When NaI in combination with chloroamine T is used, the second step is preferably performed at elevated temperatures, such as at temperatures from 40 to 80° C.

The compounds of formulae (4) and (1) can be isolated by methods known in the art, such as column chromatography.

The compound of formula (2) can be obtained by methods known in the art, for example as described in the subsection titled “Synthesis” of F. Würthner, Chem. Commun., 2004, 1564-1579.

Also part of the present invention is an electronic device comprising the compound of formula (1) as semiconducting material. Preferably, the electronic device is an organic field effect transistor (OFET).

Usually, an organic field effect transistor comprises a dielectric layer, a semiconducting layer and a substrate. In addition, an organic field effect transistor usually comprises a gate electrode and source/drain electrodes.

An organic field effect transistor can have various designs.

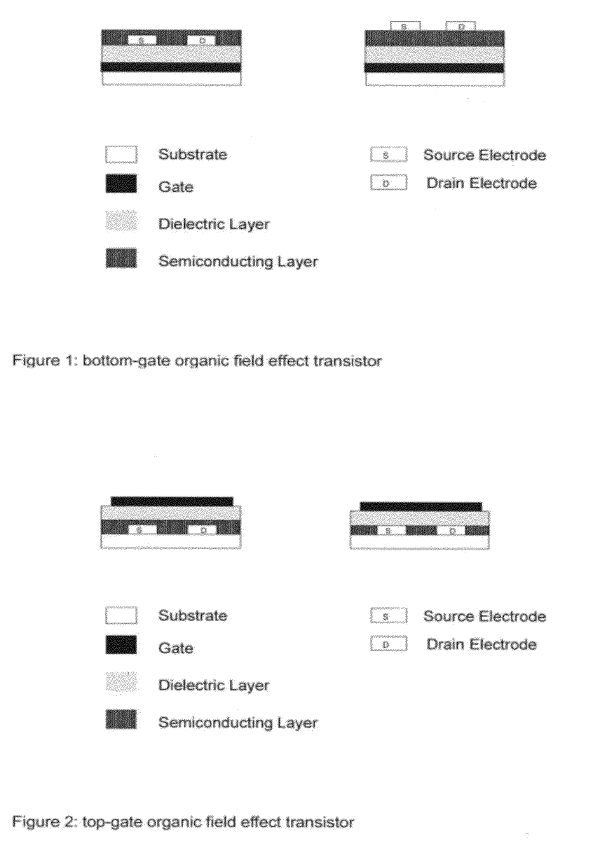

The most common design of an organic field-effect transistor is the bottom-gate design. Examples of bottom-gate designs are shown in FIG. 1.

Another design of an organic field-effect transistor is the top-gate design. Examples of top-gate designs are shown in FIG. 2.

The semiconducting layer comprises the semiconducting material of the present invention. The semiconducting layer can have a thickness of 5 to 500 nm, preferably of 10 to 100 nm, more preferably of 20 to 50 nm.

The dielectric layer comprises a dielectric material. The dielectric material can be silicon dioxide, or, an organic polymer such as polystyrene (PS), poly(methylmethacrylate) (PMMA), poly(4-vinylphenol) (PVP), poly(vinyl alcohol) (PVA), benzocyclobutene (BCB), or polyimide (PI).

The dielectric layer can have a thickness of 10 to 2000 nm, preferably of 50 to 1000 nm, more preferably of 100 to 800 nm.

The source/drain electrodes can be made from any suitable source/drain material, for example gold (Au) or tantalum (Ta). The source/drain electrodes can have a thickness of 1 to 100 nm, preferably from 5 to 50 nm.

The gate electrode can be made from any suitable gate material such as highly doped silicon, aluminium (Al), tungsten (W), indium tin oxide, gold (Au) and/or tantalum (Ta). The gate electrode can have a thickness of 1 to 200 nm, preferably from 5 to 100 nm.

The substrate can be any suitable substrate such as glass, or a plastic substrate such as polyethersulfone, polycarbonate, polysulfone, polyethylene terephthalate (PET) and polyethylene naphthalate (PEN). Depending on the design of the organic field effect transistor, a combination of the gate electrode and the dielectric layer can also function as substrate.

The organic field effect transistor can be prepared by methods known in the art.

For example, a bottom-gate organic field effect transistor can be prepared as follows: The gate electrode can be formed by depositing the gate material, for example highly doped silicon, on one side of the dielectric layer made of a suitable dielectric material, for example silicium dioxide. The other side of the dielectric layer can be optionally treated with a suitable reagent, for example with hexamethyldisilazane (HMDS). Source/drain electrodes can be deposited on this side (the side which is optionally treated with a suitable reagent) of the dielectric layer for example by vapour deposition of a suitable source/drain material, for example tantalum (Ta) and/or gold (Au). The source/drain electrodes can then be covered with the semiconducting layer by solution processing, for example drop coating, a solution of the semiconducting material of the present invention in s suitable solvent, for example in chloroform.

Also part of the invention is the use of the compound of formula (1) as semiconducting material.

In FIG. 1 two designs of a bottom-gate organic field effect transistor are shown.

In FIG. 2 two designs of a top-gate organic field effect transistor are shown.

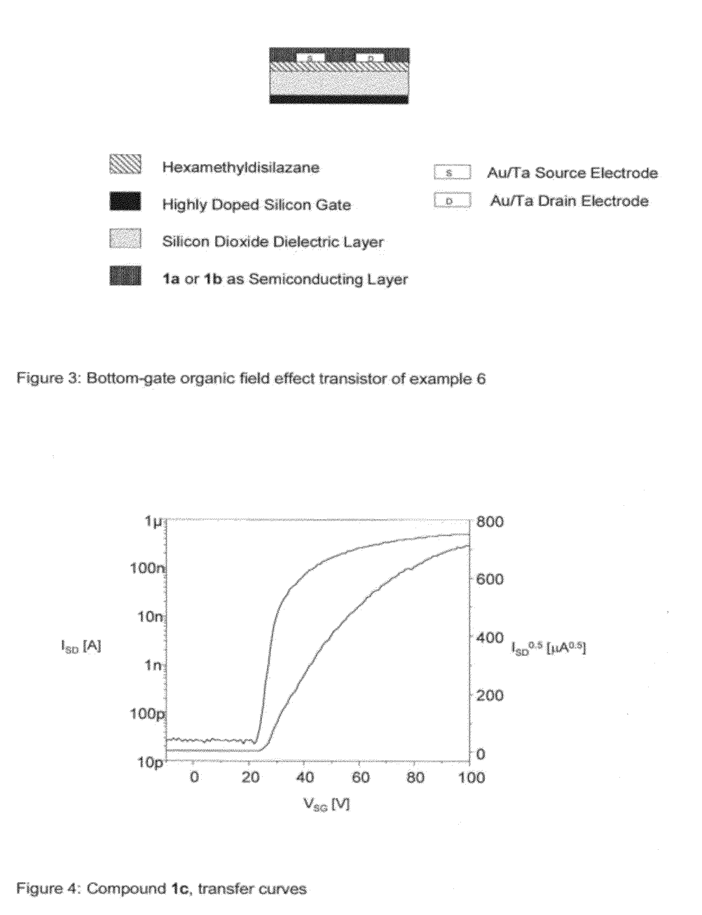

In FIG. 3 the bottom-gate organic field effect transistor of example 6 is shown.

In FIG. 4 the drain current I_{SD} [A] in relation to the gate voltage V_{SG} [V] (top transfer curve) and the drain current I_{SD}^{0.5} [μA^{0.5}] in relation to the gate voltage V_{SG} [V] (bottom transfer curve) for the bottom-gate organic field effect transistor of example 6 comprising compound 1c as semiconducting material at a drain voltage V_{SD} of 100 V is shown.

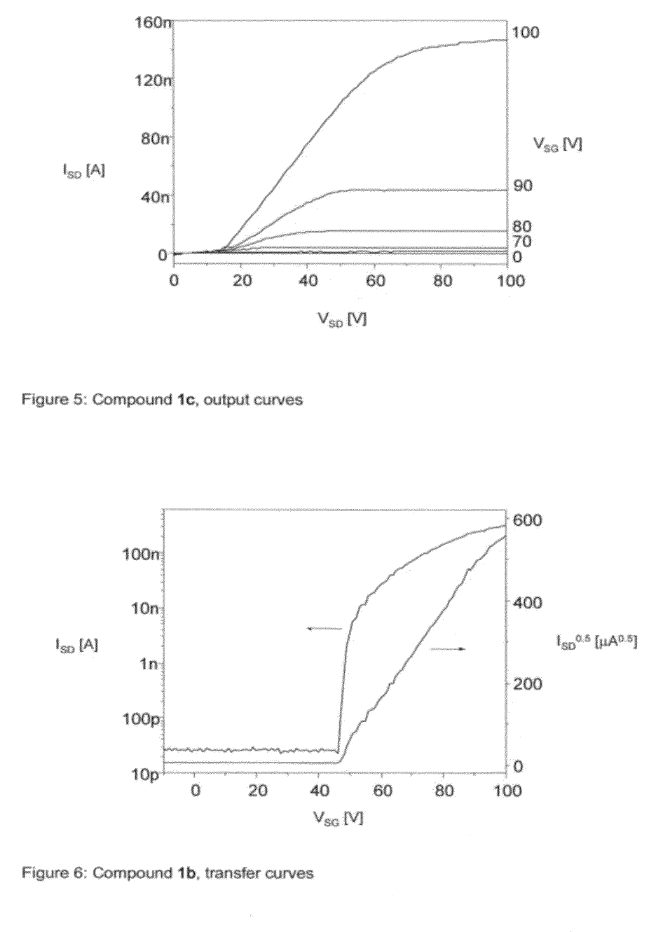

In FIG. 5 the drain current I_{SD} [A] in relation to the drain voltage V_{SD} [V] (output curve) for the bottom-gate organic field effect transistor of example 6 comprising compound 1c as semiconducting material at a gate voltage V_{SG} of 100 V (first and top curve), 90 V (second curve), 80 V (third curve), 70 V (fourth curve) and 0 V (fifth and bottom curve) is shown.

In FIG. 6 the drain current I_{SD} [A] in relation to the gate voltage V_{SG} [V] (top transfer curve) and the drain current I_{SD [μA}^{0.5}] in relation to the gate voltage V_{SG} [V] (bottom transfer curve) for the bottom-contact organic field effect transistor of example 6 comprising compound 1b as semiconducting material at a drain voltage V_{SD} of 100 V is shown.

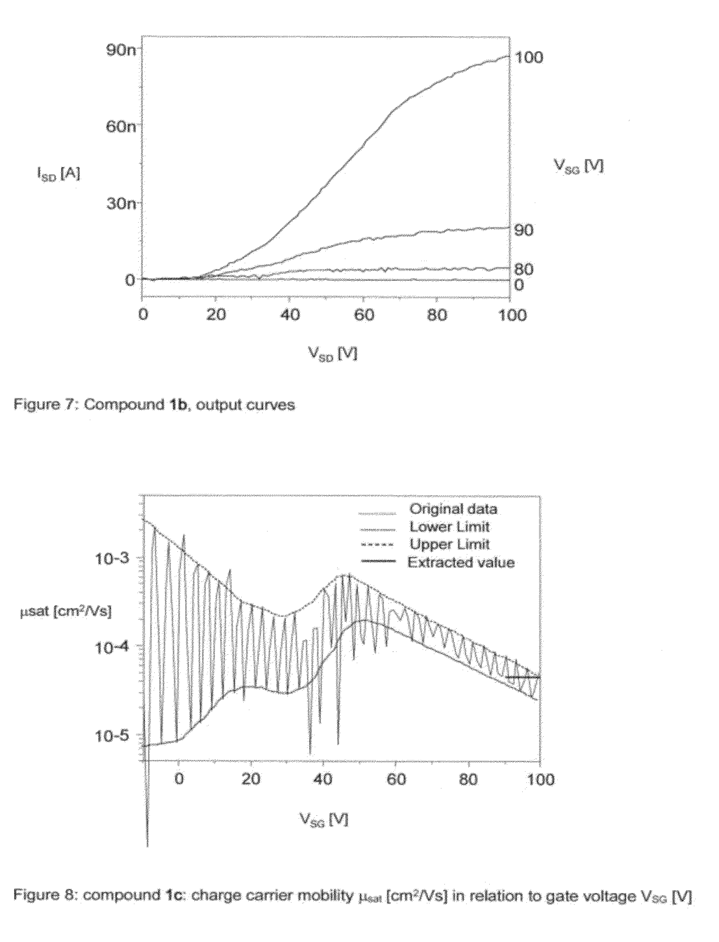

In FIG. 7 the drain current I_{SD} [A] in relation to the drain voltage V_{SD} [V] (output curve) for the bottom-gate organic field effect transistor of example 6 comprising compound 1b as semiconducting material at a gate voltage V_{SG} of 100 V (first and top curve), 90 V (second curve), 80 V (third curve), 0 V (fourth and bottom curve) is shown.

In FIG. 8 the charge carrier mobility μₛₐₜ [cm²/Vs] in relation to the gate voltage V_{SG} [V] for the bottom-gate organic field effect transistor of example 6 comprising compound 1c as semiconducting material is shown.

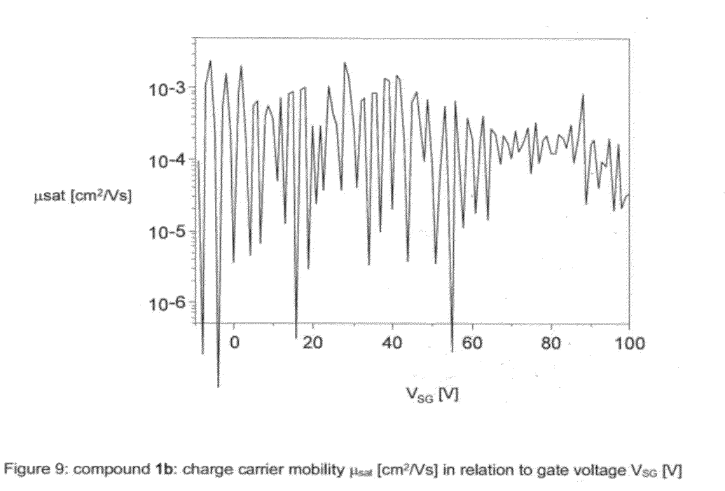

In FIG. 9 the charge carrier mobility μₛₐₜ [cm²/Vs] in relation to the gate voltage V_{SG} [V] for the bottom-gate organic field effect transistor of example 6 comprising compound 1b as semiconducting material is shown.

The advantage of the semiconducting materials of the present invention is the high solubility of these materials in solvents suitable for solution processing. In addition the semiconducting materials of the present invention show acceptable to high charge carrier mobility. In addition, the semiconducting materials are stable, in particular towards oxidation, under ambient conditions.

### EXAMPLES

### Example 1

### Preparation of N,N′-bis(1-ethylpropyl)-2,5,8,11-tetrakis[4,4,5,5-tetramethyl-1,3,2-dioxaborolan-2-yl]perylene-3,4:9,10-tetracarboxylic acid bisimide (4a)

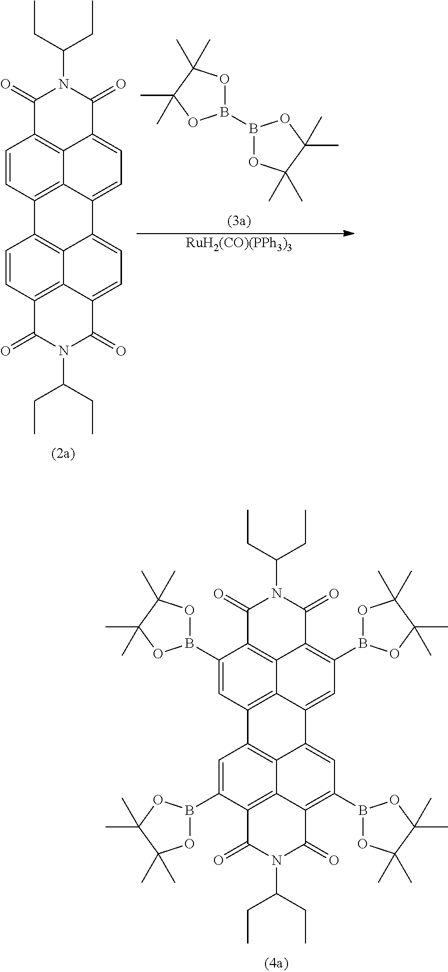

N,N′-Bis(1-ethylpropyl) perylene-3,4:9,10-tetracarboxylic acid bisimide (2a) (100 mg, 0.189 mmol) and bispinacolonediboronate (3a) (0.383 g, 1.51 mmol) are mixed together and dissolved in 2 mL dry mesitylene and 0.15 mL dry pinacolone. Argon is bubbled trough the solution for 30 minutes. RuH₂(CO)(PPh₃)₃ (0.082 mg, 0.09 mmol) is added to the reaction mixture and the vessel heated to 140° C. for 30 hours. After cooling the system to room temperature, the solvent is evaporated and the desired compound purified by column chromatography (silica, CH₂Cl₂/AcOEt 50/1). An orange bright solid is obtained with 60% yield (117 mg, 0.113 mmol).

¹H NMR (250 MHz, CD₂Cl₂) δ 8.59 (s, J=7.3 Hz, 4H), 4.94 (tt, J=9.2, 6.0 Hz, 2H), 2.33-2.10 (m, 4H), 2.04-1.84 (m, 4H), 1.51 (s, J=7.2 Hz, 48H), 0.92 (t, J=7.4 Hz, 12H). FD Mass Spectrum (8 kV): m/z=1033.33 (100%) [M+]. Absorption: 537 nm (in toluene). Emission: 548 nm (in toluene, exc 537 nm). Extinction Coefficient: 7.30×10⁴M⁻¹cm⁻¹. Fluorescence Quantum Yield: 0.89. Elemental Analysis: theoretical: C: 67.34%; H: 7.21%; N: 2.71%; experimental: C: 67.29%; H: 7.40%; N: 2.96%.

### Example 2

### Preparation of N,N′-Bis(1-ethylpropyl)-2,5,8,11-tetrabromo-perylene-3,4:9,10-tetracarboxylic acid bisimide (1a)

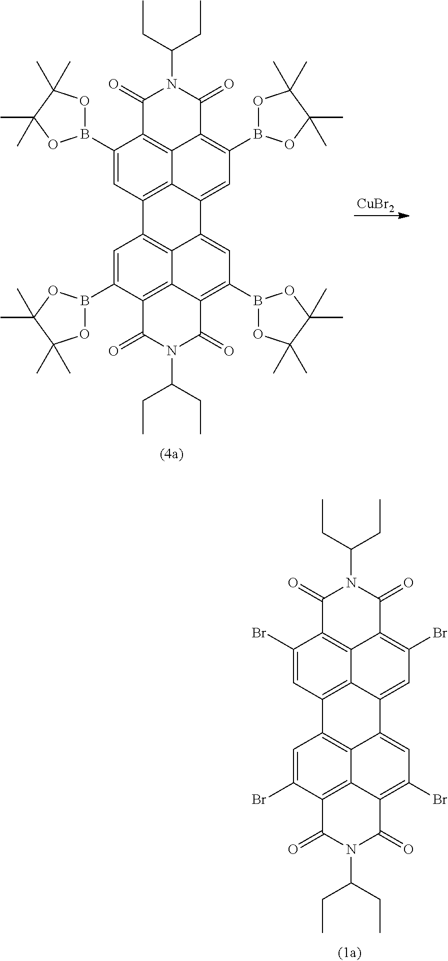

N,N′-Bis(1-ethylpropyl)-2,5,8,11-tetrakis[4,4,5,5-tetramethyl-1,3,2-dioxaborolan-2-y]perylene-3,4:9,10-tetracarboxylic acid bisimide (4a), prepared as described in example 1, (400 mg, 0.387 mmol) and copper(II) bromide (1.73 g, 7.73 mmol) are suspended in a mixture of methanol (3 mL) and water (3 mL) and heated at 100° C. for 6 hours. The reaction mixture is then poured in water and extracted with dichloromethane. The organic phase is dried over magnesium sulfate and the solvent evaporated. The compound la is obtained as an orange solid after column chromatography (silica, dichloromethane) in 90% yield (295 mg, 0.39 mmol).

¹H NMR (250 MHz, CD₂Cl₂) δ 8.71 (s, 4H), 4.95 (m, 2H), 2.23-2.02 (m, 4H), 1.97-1.78 (m, 4H), 0.84 (t, J=7.5 Hz, 12H). FD Mass Spectrum (8 kV): m/z=844.8 (100%) [M+].

### Example 3

### Preparation of N,N′-bis(1-heptyloctyl)-2,5,8,11-tetrakis[4,4,5,5-tetramethyl-1,3,2-dioxaborolan-2-yl]perylene-3,4:9,10-tetracarboxylic acid bisimide (4b)

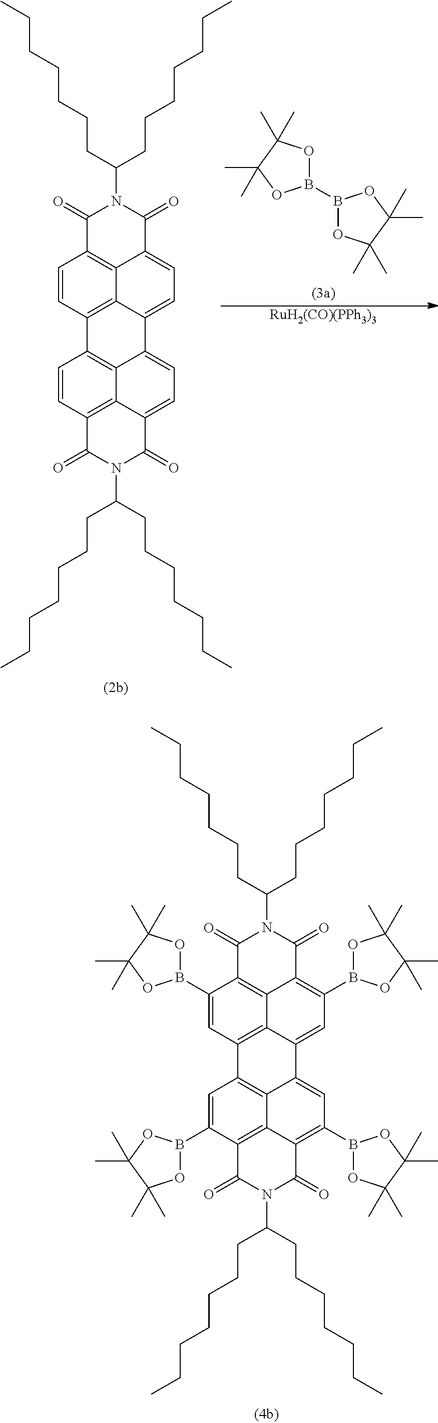

N,N′-Bis(1-heptyloctyl)perylene-3,4:9,10-tetracarboxylic acid bisimide (2b) (100 mg, 0.12 mmol) and bispinacolonediboronate (3a) (250 mg, 0.99 mmol) are mixed together and dissolved in 1 mL anhydrous mesitylene and 1 mL anhydrous pinacolone. Argon is bubbled through the solution for 30 minutes. RuH₂(CO)(PPh₃)₃ (23 mg, 0,03 mmol) is added to the mixture and the reaction mixture is heated at 140° C. for 30 hours. After cooling the system to room temperature, the solvent is evaporated and the desired compound purified by column chromatography (CH₂Cl₂). 4b is obtained as a red solid in 70% yield (113 mg, 0.09 mmol).

¹H NMR (250 MHz, CD₂Cl₂) δ 8.58 (s, 4H), 5.06 (s, 2H), 2.35-2.06 (m, 4H), 1.98-1.72 (m, 4H), 1.50 (s, 48H), 1.24 (s, 40H), 0.84 (t, J=6.5 Hz, 12H). ¹³C NMR (126 MHz, CD₂Cl₂) δ 166.27 (d, J=98.5 Hz), 139.79-138.86 (m), 133.80 (s), 128.82 (s), 127.57 (d, J=69.0 Hz), 127.30 (s), 126.29 (s), 84.90 (s), 55.19 (s), 32.83 (s), 32.45 (s), 30.03 (s), 29.76 (s), 27.37 (s), 25.38 (s), 23.22 (s), 14.43 (s). FD/MS (8 kV): m/z=1312.4 (100%) [M+]. UV-Vis (in toluene): λₘₐₓ (ε[M⁻¹cm⁻¹]): 538 nm (5.57×10⁴). Fluorescence (in toluene, λₑₓ=538 nm): 548 nm. Φ_{F}: 0.83. Elem. Anal.: theoretical: C: 71.24%; H: 8.74%; N: 2.13%; experimental: C: 70.76%; H: 8.27%; N: 2.50%.

### Example 4

### Preparation of N,N′-Bis(1-heptyloctyI)-2,5,8,11-tetrachloro-perylene-3,4:9,10-tetracarboxylic acid bisimide (1b)

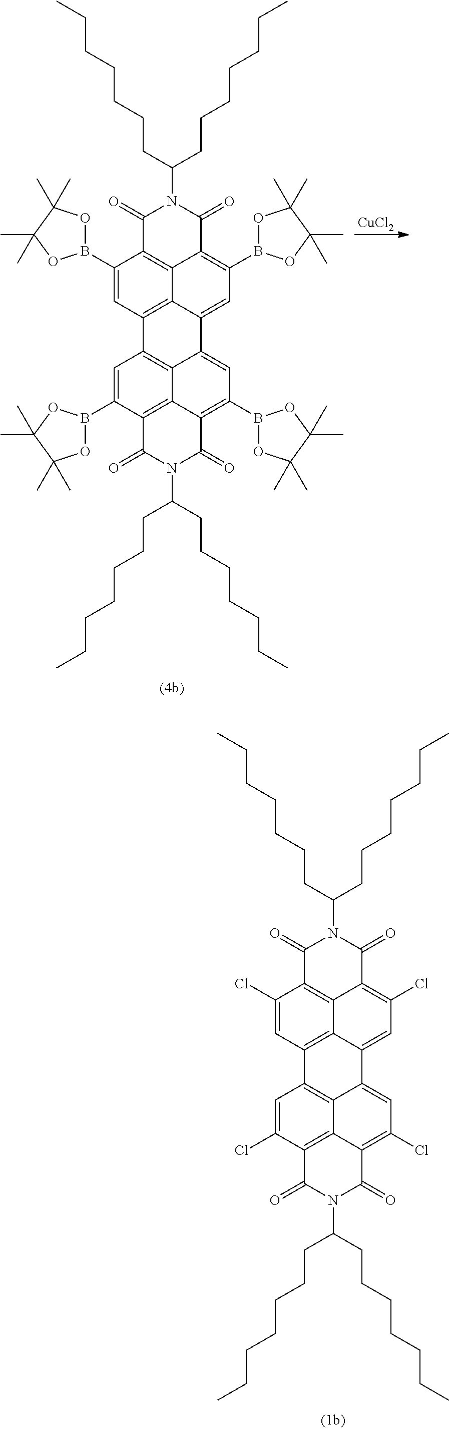

N,N′-Bis(1-heptyloctyl)-2,5,8,11-tetrakis[4,4,5,5-tetramethyl-1,3,2-dioxaborolan-2-y]perylene-3,4:9,10-tetracarboxylic acid bisimide (4b), prepared as described in example 3, (1.00 g, 0.76 mmol) and copper(II) chloride (1.23 g, 9.13 mmol) are suspended in a mixture of methanol (3 mL) and water (3 mL) and heated in a closed vessel at 100° C. for 6 hours. The reaction mixture is then poured in water and extracted with dichloromethane. The organic phase is dried over magnesium sulfate and the solvent evaporated. The compound 1b is obtained as an orange solid after column chromatography (silica, dichloromethane) in 87% yield (0.628 g, 0.66 mmol).

¹H NMR (250 MHz, CD₂Cl₂) δ 8.43 (s, 4H), 5.06 (m, 2H), 2.22-1.99 (m, 4H), 1.79 (m, 4H), 1.20 (m, 40H), 0.82-0.69 (m, 12H). FD Mass Spectrum (8 kV): m/z=947.7 (100%) [M+].

### Example 5

### Preparation of N,N′-Bis(1-heptyloctyI)-2,5,8,11-tetrabromo-perylene-3,4:9,10-tetracarboxylic acid bisimide (1c)

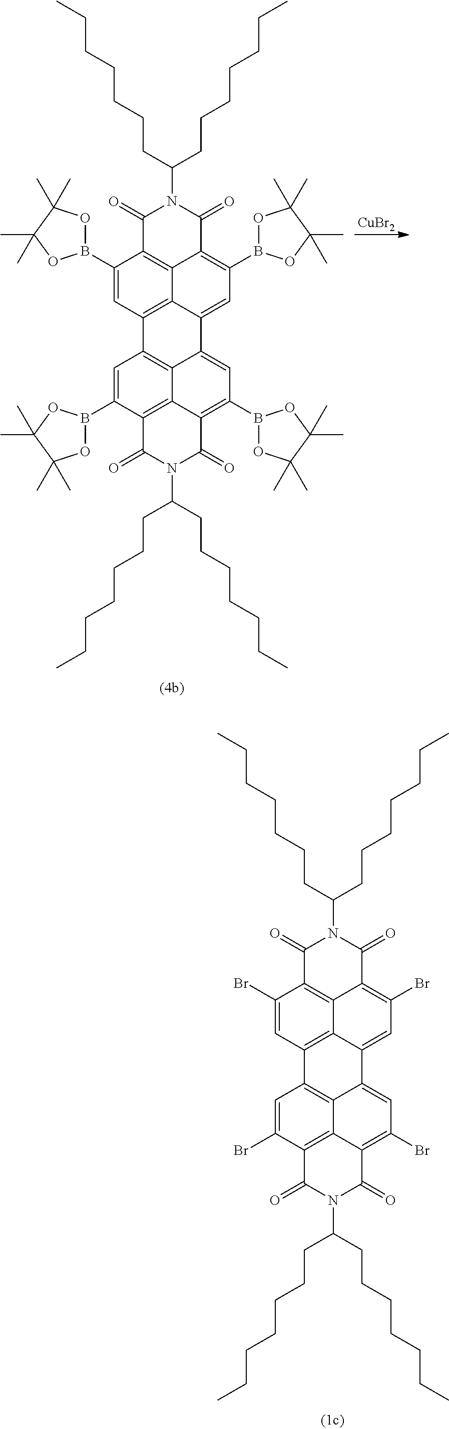

N,N′-Bis(1-heptyloctyl)-2,5,8,11-tetrakis[4,4,5,5-tetramethyl-1,3,2-dioxaborolan-2-y]perylene-3,4:9,10-tetracarboxylic acid bisimide (4b), prepared as described in example 3, (1.00 g, 0.76 mmol) and copper(II)-bromide (3.39 g, 15.20 mmol) are suspended in 80 mL of a 1/1/1 mixture of dioxane/methanol/water and heated at 120° C. for 12 hours. The reaction mixture is then cooled, poured in water and extracted with dichloromethane. The organic phase is dried over magnesium sulfate and the solvent is evaporated. Compound 1c is obtained as an orange solid after column chromatography (silica, dichloromethane: petrol ether 1:1) in 92% yield (0.79 g, 0.70 mmol).

¹H NMR (500 MHz, CD₂Cl₂) δ 8.74 (s, 4H), 5.22-5.07 (m, 2H), 2.19 (m, 4H), 1.88 (m, 4H), 1.39-1.18 (m, 40H), 0.84 (t, J=6.3 Hz, 12H). ¹³C NMR (126 MHz, CD₂Cl₂) δ 161.65 (s), 133.11 (s), 132.76 (s), 132.05 (s), 129.36 (s), 125.15 (s), 122.07 (s), 56.26 (s), 32.80 (s), 32.37 (s), 30.03 (s), 29.78 (s), 27.52 (s), 23.20 (s), 14.41 (s). FD/MS (8 kV): m/z=1124.8 (100%) [M+]. UV-Vis (in dichloromethane): λₘₐₓ (ε[M⁻¹cm⁻¹]): 509 nm (7.9×10⁴). Fluorescence (in dichlorornethane, λ=509 nm): 519 nm. Φ_{F}: 0.21. Elem. Anal.: theoretical: C: 57.56%; H: 5.90%; N: 2.49%; experimental: C: 57.25%; H: 6.27%; N: 2.52%.

### Example 6

### Preparation of Bottom-Gate Organic Field Effect Transistors Containing Compound 1b, Respectively, 1c as Semiconducting Material

Thermally grown silicon dioxide (thickness: 200 nm) is used as dielectric layer. The gate electrode is formed by depositing highly doped silicon on one side of the dielectric layer. The other side of the dielectric layer is treated with hexamethyldisilazane (HMDS) by vapour deposition of hexamethyldisilazane. The contact angle of the surface of the HMPS-treated side of the dielectric layer is 93.2±1.3°. Source/drain electrodes (Ta (thickness: 10 nm) covered by Au (thickness: 40 nm)) are deposited on the HMPS-treated side of the dielectric layer by vapour deposition. The channel length is 20 μm and the channel width is 1.4 mm, affording W/L=70. The source/drain electrodes are then covered with the semiconducting layer (thickness: ca. 100 nm) by drop-casting a solution of compound 1b, respectively, 1c in chloroform (concentration=10 mg/mL) in a nitrogen filled glove box (O₂ content: 0.1 ppm, H₂O content: 0.0 ppm, pressure: 1120 Pa, temperature: 17° C.) using a Keithley 4200 machine.

The design of the bottom-gate organic field effect transistor of example 6 is shown in FIG. 3.

The drain current I_{SD} [A] in relation to the gate voltage V_{SG} [V] (top transfer curve) and the drain current I_{SD}^{0.5} [μA^{0.5}] in relation to the gate voltage V_{SG} [V] (bottom transfer curve) for the bottom-gate organic field effect transistor of example 6 comprising compound 1c as semiconducting material at a drain voltage V_{SD} of 100 V is determined in a nitrogen filled glove box (O₂ content: 0.1 ppm, H₂O content: 0.0 ppm, pressure: 1120 Pa, temperature: 17° C.) using a Keithley 4200 machine is shown. The results are shown in FIG. 4.

The drain current I_{SD} in relation to the drain voltage V_{SD} (output curve) for the bottom-gate organic field effect transistor of example 6 comprising compound 1c as semiconducting material at a gate voltage V_{SG} of 100 V (first and top curve), 90 V (second curve), 80 V (third curve), 70 V (fourth curve) and 0 V (fifth and bottom curve) is determined in a nitrogen filled glove box (O₂ content: 0.1 ppm, H₂O content: 0.0 ppm, pressure: 1120 Pa, temperature: 17° C.) using a Keithley 4200 machine is shown. The results are shown in FIG. 5.

The drain current I_{SD} [A] in relation to the gate voltage V_{SG} [V] (top transfer curve) and the drain current I_{SD}^{0.5} [μA^{0.5}] in relation to the gate voltage V_{SG} [V] (bottom transfer curve) for the bottom-gate, organic field effect transistor of example 6 comprising compound 1b as semiconducting material at a drain voltage V_{SD} of 100 V is determined in a nitrogen filled glove box (O₂ content: 0.1 ppm, H₂O content: 0.0 ppm, pressure: 1120 Pa, temperature: 17° C.) using a Keithley 4200 machine is shown. The results are shown in FIG. 6.

The drain current I_{SD} in relation to the drain voltage V_{SD} (output curve) for the bottom-gate organic field effect transistor of example 6 comprising compound 1b as semiconducting material at a gate voltage V_{SG} of 100 V (first and top curve), 90 V (second curve), 80 V (third curve) and 0 V (fourth and bottom curve) is determined in a nitrogen filled glove box (O₂ content: 0.1 ppm, H₂O content: 0.0 ppm, pressure: 1120 Pa, temperature: 17° C.) using a Keithley 4200 machine is shown. The results are shown in FIG. 7.

In FIG. 8 the charge carrier mobility μₛₐₜ [cm²/Vs] in relation to the gate voltage V_{SG} [V] for the bottom-gate organic field effect transistor of example 6 comprising compound 1c as semiconducting material is shown.

In FIG. 9 the charge carrier mobility μₛₐₜ [cm²/Vs] in relation to the gate voltage V_{SG} [V] for the bottom-gate organic field effect transistor of example 6 comprising compound 1b as semiconducting material is shown.

The average values and the 90% confidence interval (in parentheses) of the charge carrier mobilities μₛₐₜ [cm²/Vs], the I_{ON}/I_{OFF} ratios and the switch-on voltages V_{SO} [V] for the bottom-gate organic field effect transistors of example 6 comprising compound 1b, respectively, 1c, as semiconducting material are given in table 1. The switch-on voltage V_{SO} [V] is the gate voltage V_{SG} [V] where the drain current I_{SD} [A] starts to increase (out of the off-state).

TABLE 1

| | μₛₐₜ | | V_{SO} |
|---|---|---|---|
| Compound | [cm²/Vs] | I_{ON}/I_{OFF} | [V] |
| 1c | 4.5 (±2.5) × 10⁻⁵ | 1.5 (±1.2) × 10⁴ | 40.0 (±12.5) |
| 1b | 4.0 (±3.7) × 10⁻⁵ | 5.2 (±4.1) × 10³ | 54.5 (±10.7) |

### Example 7

### Preparation of N,N′-Bis(1-heptyloctyl)-2,5,8,11-tetraiodo-perylene-3,4:9,10-tetracarboxylic acid bisimide (1d)

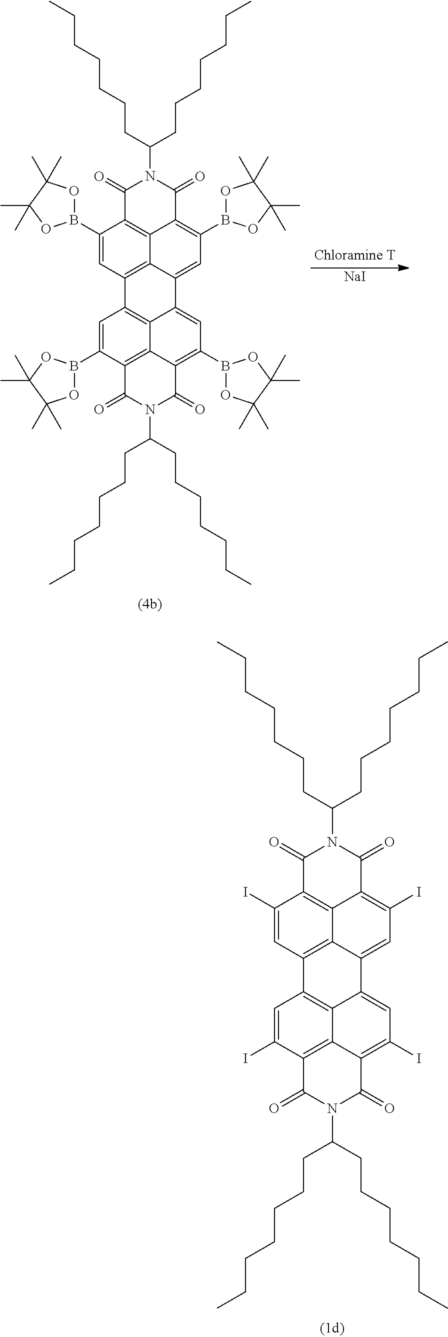

N,N′-Bis(1-heptyloctyl)-2,5,8,11-tetrakis[4,4,5,5-tetramethyl-1,3,2-dioxaborolan-2-y]perylene-3,4:9,10-tetracarboxylic acid bisimide (4b), prepared as described in example 3, (100 mg, 0.08 mmol) is suspended in a mixture of 1/1 water/THF (50 mL). Chloramine T (600 mg, 4.53 mmol) and sodium iodide (680 mg, 4.53 mmol) are added to the mixture. The vessel is sealed and heated at 55° C. for 12 hours without light. After cooling the reaction mixture to room temperature, saturated solution of sodium sulfite (10 mL) is added. Successively, the reaction mixture is added to water (100 mL). The solid is filtrated, dried and purified by column chromatography (1/1 petrol ether, dichlromethane). The compound 1d is obtained as a red solid in 42% yield.

¹H NMR (700 MHz, CD₂Cl₂) δ 9.10 (s, 4H), 5.22-5.11 (m, 2H), 2.26-2.14 (m, 4H), 1.95-1.87 (m, 4H), 1.40-1.18 (m, 40H), 0.84 (t, J=7.0 Hz, 12H). ¹³C NMR (176 MHz, CD₂Cl₂) δ 161.27 (s), 139.06 (s), 138.60 (s), 132.38 (s), 131.73 (s), 126.28 (s), 124.09 (s), 56.56 (s), 32.76 (s), 32.38 (s), 30.04 (s), 29.80 (s), 27.52 (s), 32.22 (s), 14.43 (s). FD/MS (8 kV): m/z=1315.4 (100%) [M+]. UV-VIS (in dichloromethane): λₘₐₓ (ε[M⁻¹cm⁻¹]): 518 nm (7.23×10⁴). Elem. Anal.: theoretical: C: 49.33%; H: 5.06%; N: 2.13%; experimental: C: 49.68%; H: 5.01%; N: 2.24%.

### Example 8

### Preparation of N,N′-Bis-octyl-2,5,8,11-tetrabromo-perylene-3,4:9,10-tetracarboxylic acid bisimide (1e)

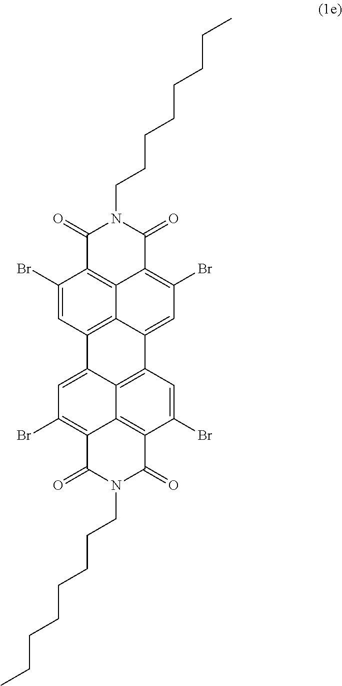

N,N′-Bis-octyl-2,5,8,11-tetrakis[4,4,5,5-tetramethyl-1,3,2-dioxaborolan-2-y]perylene-3,4:9,10-tetracarboxylic acid bisimide (0.68 mg, 0.61 mmol) and copper(II) bromide (1.62 g, 7.3 mmol) are suspended in a mixture of dioxane (10 mL), methanol (3 ml) and water (3 ml) and heated at 120° C. for 12 hours. The reaction mixture is then poured into HCl (1.0 M) and the solid so obtained filtered. The desired compound is obtained as an orange solid after column chromatography (silica, dichloromethane) in 30% yield (0.17 mg, 0.18 mmol).

¹H NMR (250 MHz, THF-d8) δ=9.02 (s, 4H), 4.20 (m, 4H), 1.42 (m,24H), 0.95 (t, J=6.0, 6H). FD Mass Spectrum (8 kV): m/z=932.6 (100%) [M+].

### Example 9

### Preparation of N,N′-Bis-(2-ethylhexyl)-2,5,8,11-tetrabromo-perylene-3,4:9,10-tetracarboxylic acid bisimide (1f)

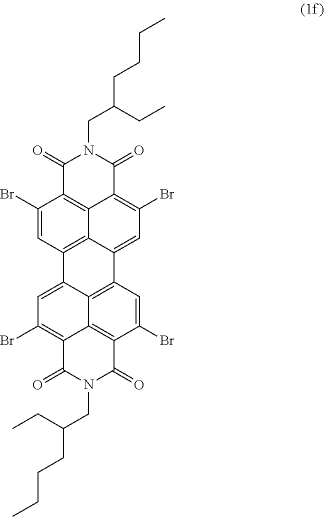

N,N′-Bis-(2-ethylhexyl)-2,5,8,11-tetrakis[4,4,5,5-tetramethyl-1,3,2-dioxaborolan-2-y]perylene-3,4:9,10-tetracarboxylic acid bisimide (0.68 mg, 0.61 mmol) and copper(II) bromide (1.62 g, 7.3 mmol) are suspended in a mixture of dioxane (10 mL), methanol (3 ml) and water (3 ml) and heated at 120° C. for 12 hours. The reaction mixture is then poured into HCl (1.0 M) and the solid so obtained filtered. The desired compound is obtained as an orange solid after column chromatography (silica, dichloromethane) in 39% yield (0.22 mg, 0.24 mmol).

¹H NMR (250 MHz, Methylene Chloride-d2) δ=8.56 (s,4H), 4.06 (m, 4H),2.16 (m, 2H), 1.17 (m, 16H), 0.82 (m, 12H). FD Mass Spectrum (8 kV): m/z=932.8 (100%) [M+].

1. A compound of formula 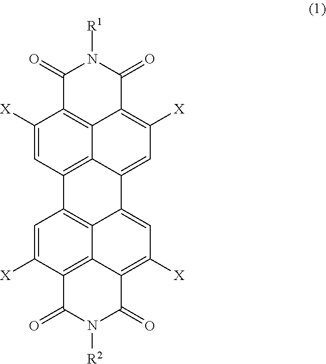
wherein
R¹ and R² are independently from each other selected from the group consisting of H, C₁₋₃₀-alkyl optionally substituted with 1 to 30 substituents R^{a}, C₂₋₃₀-alkenyl optionally substituted with 1 to 30 substituents R^{a}, C₂₋₃₀-alkynyl optionally substituted with 1 to 30 substituents R^{a}, C₃₋₁₀-cycloalkyl optionally substituted with 1 to 10 substituents R^{b}, C₅₋₁₀-cycloalkenyl optionally substituted with 1 to 10 substituents R^{b}, 3-14 membered cycloheteroalkyl optionally substituted with 1 to 8 substituents R^{b}, C₆₋₁₄-aryl optionally substituted with 1 to 8 substituents R^{c} and 5-14 membered heteroaryl optionally substituted with 1 to 8 substituents R^{c},
wherein
R^{a} at each occurrence are independently from each other selected from the group consisting of halogen, —CN, —NO₂, —N₃, —OH, C₁₋₃₀-alkoxy optionally substituted with 1 to 6 substituents Rⁱ, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl optionally substituted with 1 to 30 substituents Rⁱ, —SO₂—C₁₋₃₀-alkyl optionally substituted with 1 to 30 substituents Rⁱ, —NH₂, —NHR³, —NR³R⁴, —[NR³R⁴R⁵]⁺, —NH—COR³, —COOH, —COOR³, —CONH₂, —CONHR³, —CONR³R⁴, —CO—H, —COR³, C₃₋₁₀-cycloalkyl optionally substituted with 1 to 10 substituents Rⁱⁱ, C₅₋₁₀-cycloalkenyl optionally substituted with 1 to 10 substituents Rⁱⁱ, 3-14 membered cycloheteroalkyl optionally substituted with 1 to 10 substituents Rⁱⁱ, C₆₋₁₄-aryl optionally substituted with 1 to 8 substituents Rⁱⁱⁱ and 5-14 membered heteroaryl optionally substituted with 1 to 8 substituents Rⁱⁱⁱ;
R^{b} at each occurrence are independently from each other selected from the group consisting of halogen, —CN, —NO₂, —OH, C₁₋₃₀-alkoxy optionally substituted with 1 to 30 substituents Rⁱ, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl optionally substituted with 1 to 30 substituents Rⁱ, —NH₂, —NHR³, —NR³R⁴, —[NR³R⁴R⁵]⁺, —NH—COR³, —COOH, —COOR³, —CONH₂, —CONHR³, —CONR³R⁴, —CO—H, —COR³, C₁₋₃₀-alkyl optionally substituted with 1 to 30 substituents Rⁱ, C₂₋₃₀-alkenyl optionally substituted with 1 to 30 substituents Rⁱ, C₂₋₃₀-alkynyl optionally substituted with 1 to 30 substituents R¹, C₃-10-cycloalkyl optionally substituted with 1 to 10 substituents Rⁱⁱ, C₅₋₁₀-cycloalkenyl optionally substituted with 1 to 10 substituents Rⁱⁱ, 3-14 membered cycloheteroalkyl optionally substituted with 1 to 10 substituents Rⁱⁱ, C₆₋₁₄-aryl optionally substituted with 1 to 8 substituents Rⁱⁱⁱ and 5-14 membered heteroaryl optionally substituted with 1 to 8 substituents Rⁱⁱⁱ;
R^{c} at each occurrence are independently from each other selected from the group consisting of halogen, —CN, —NO₂, —N₃, —OH, C₁₋₃₀-alkoxy optionally substituted with 1 to 30 substituents Rⁱ, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl optionally substituted with 1 to 30 substituents Rⁱ, —SO₂—C₁₋₃₀-alkyl optionally substituted with 1 to 30 substituents Rⁱ, —NH₂, —NHR³, —NR³R⁴, —[NR³R⁴R⁵]⁺, —NH—COR³, —COOH, —COOR³, —CONH₂, —CONHR³, —CONR³R⁴, —CO—H, —COR³, C₁₋₃₀-alkyl optionally substituted with 1 to 30 substituents Rⁱ, C₂₋₃₀-alkenyl optionally substituted with 1 to 30 substituents Rⁱ, C₂₋₃₀-alkynyl optionally substituted with 1 to 30 substituents Rⁱ, C₃₋₁₀-cycloalkyl optionally substituted with 1 to 10 substituents Rⁱⁱ, C₅₋₁₀-cycloalkenyl optionally substituted with 1 to 10 substituents Rⁱⁱ, 3-14 membered cycloheteroalkyl optionally substituted with 1 to 10 substituents Rⁱⁱ, C₆₋₁₄-aryl optionally substituted with 1 to 8 substituents Rⁱⁱⁱ and 5-14 membered heteroaryl optionally substituted with 1 to 8 substituents Rⁱⁱⁱ;
wherein
R³, R⁴ and R⁵ at each occurrence are independently from each other selected from the group consisting of C₁₋₃₀-alkyl optionally substituted with 1 to 30 substituents Rⁱ, C₂₋₃₀-alkenyl optionally substituted with 1 to 30 substituents Rⁱ, C₂₋₃₀-alkynyl optionally substituted with 1 to 30 substituents Rⁱ, C₃₋₁₀-cycloalkyl optionally substituted with 1 to 10 substituents Rⁱⁱ, C₅₋₁₀-cycloalkenyl optionally substituted with 1 to 10 substituents Rⁱⁱ, 3-14 membered cycloheteroalkyl optionally substituted with 1 to 10 substituents Rⁱⁱ, C₆₋₁₄-aryl optionally substituted with 1 to 8 substituents Rⁱⁱⁱ and 5-14 membered heteroaryl optionally substituted with 1 to 8 substituents Rⁱⁱⁱ,
Rⁱ at each occurrence are independently from each other selected from the group consisting of halogen, —CN, —NO₂, —N₃, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl, —SO₂—C₁₋₃₀-alkyl, —NH₂, —NHR⁶, —NR⁶R⁷, —[NR⁶R⁷R⁸]⁺, —NH—COR⁶, —COOH, —COOR⁶, —CONH₂, —CONHR⁶, —CONR⁶R⁷, —CO—H, —COR⁶, C₃₋₁₀-cycloalkyl, C₅₋₁₀-cycloalkenyl, 3-14 membered cycloheteroalkyl, C₆₋₁₄-aryl and 5-14 membered heteroaryl,
Rⁱⁱ at each occurrence are independently from each other selected from the group consisting of halogen, —CN, —NO₂, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR⁶, —S—C₁₋₃₀-alkyl, —NH₂, —NHR⁶, —NR⁶R⁷, —[NR⁶R⁷R⁸]⁺, —NH—COR⁶, —COOH, —COOR⁶, —CONH₂, —CONHR⁶, —CONR⁶R⁷, —CO—H, —COR⁶, C₁₋₃₀-alkyl, C₂₋₃₀-alkenyl, C₂₋₃₀-alkynyl, C₃₋₁₀-cycloalkyl, C₅₋₁₀-cycloalkenyl, 3-14 membered cycloheteroalkyl, C₆₋₁₄-aryl and 5-14 membered heteroaryl,
Rⁱⁱⁱ at each occurrence are independently from each other selected from the group consisting of halogen, —CN, —NO₂, —N₃, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR⁶, —S—C₁₋₃₀-alkyl, —SO₂—C₁₋₃₀-alkyl, —NH₂, —NHR⁶, —NR⁶R⁷, —[NR⁶R⁷R⁸]⁺, —NH—COR⁶, —COOH, —COOR⁶, —CONH₂, —CONHR⁶, —CONR⁶R⁷, —CO—H, —COR⁶, C₁₋₃₀-alkyl, C₂₋₃₀-alkenyl, C₂₋₃₀-alkynyl, C₃₋₁₀-cycloalkyl, C₅₋₁₀-cycloalkenyl, 3-14 membered cycloheteroalkyl, C₆₋₁₄-aryl and 5-14 membered heteroaryl,
wherein
R⁶, R⁷ and R⁸ at each occurrence are independently from each other selected from the group consisting of C₁₋₃₀-alkyl, C₂₋₃₀-alkenyl, C₂₋₃₀-alkynyl, C₃₋₁₀-cycloalkyl, C₅₋₁₀-cycloalkenyl, 3-14 membered cycloheteroalkyl, C₆₋₁₄-aryl and 5-14 membered heteroaryl,
and
X is —Cl, —Br or —I.

2. The compound of claim 1, wherein
R¹ and R² are independently from each other selected from the group consisting of H, C₁₋₃₀-alkyl optionally substituted with 1 to 30 substituents R^{a}, C₂₋₃₀-alkenyl optionally substituted with 1 to 30 substituents R^{a}, C₃₋₁₀-cycloalkyl optionally substituted with 1 to 10 substituents R^{b}, and C₆₋₁₄-aryl optionally substituted with 1 to 8 substituents R^{c},
wherein
R^{a} at each occurrence are independently from each other selected from the group consisting of halogen, —CN, —NO₂, —N₃, —OH, C₁₋₃₀-alkoxy optionally substituted with 1 to 6 substituents Rⁱ, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl optionally substituted with 1 to 30 substituents Rⁱ, —SO₂—C₁₋₃₀-alkyl optionally substituted with 1 to 30 substituents Rⁱ, —NH₂, —NHR³, —NR³R⁴, —[NR³R⁴R⁵]⁺, —NH—COR³, —COOH, —COOR³, —CONH₂, —CONHR³, —CONR³R⁴, —CO—H, —COR³, C₃₋₁₀-cycloalkyl optionally substituted with 1 to 10 substituents Rⁱⁱ, and C₆₋₁₄-aryl optionally substituted with 1 to 8 substituents Rⁱⁱⁱ;
R^{b} at each occurrence are independently from each other selected from the group consisting of halogen, —CN, —NO₂, —OH, C₁₋₃₀-alkoxy optionally substituted with 1 to 30 substituents Rⁱ, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl optionally substituted with 1 to 30 substituents Rⁱ, —NH₂, —NHR³, —NR³R⁴, —[NR³R⁴R⁵]⁺, —NH—COR³, —COOH, —COOR³, —CONH₂, —CONHR³, —CONR³R⁴, —CO—H, —COR³, C₁₋₃₀-alkyl optionally substituted with 1 to 30 substituents Rⁱ, C₂₋₃₀-alkenyl optionally substituted with 1 to 30 substituents Rⁱ, C₃₋₁₀-cycloalkyl optionally substituted with 1 to 10 substituents Rⁱⁱ, and C₆₋₁₄-aryl optionally substituted with 1 to 8 substituents Rⁱⁱⁱ;
R^{c} at each occurrence are independently from each other selected from the group consisting of halogen, —CN, —NO₂, —N₃, —OH, C₁₋₃₀-alkoxy optionally substituted with 1 to 30 substituents Rⁱ, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl optionally substituted with 1 to 30 substituents Rⁱ, —SO₂—C₁₋₃₀-alkyl optionally substituted with 1 to 30 substituents Rⁱ, —NH₂, —NHR³, —NR³R⁴, —[NR³R⁴R⁵]⁺, —NH—COR³, —COOH, —COOR³, —CONH₂, —CONHR³, —CONR³R⁴, —CO—H, —COR³, C₁₋₃₀-alkyl optionally substituted with 1 to 30 substituents Rⁱ, C₂₋₃₀-alkenyl optionally substituted with 1 to 30 substituents Rⁱ, C₃₋₁₀-cycloalkyl optionally substituted with 1 to 10 substituents Rⁱⁱ, and C₆₋₁₄-aryl optionally substituted with 1 to 8 substituents Rⁱⁱⁱ;
wherein
R³, R⁴ and R⁵ at each occurrence are independently from each other selected from the group consisting of C₁₋₃₀-alkyl optionally substituted with 1 to 30 substituents Rⁱ, C₂₋₃₀-alkenyl optionally substituted with 1 to 30 substituents Rⁱ, C₃₋₁₀-cycloalkyl optionally substituted with 1 to 10 substituents Rⁱⁱ, and C₆₋₁₄-aryl optionally substituted with 1 to 8 substituents Rⁱⁱⁱ,
Rⁱ at each occurrence are independently from each other selected from the group consisting of halogen, —CN, —NO₂, —N₃, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl, —SO₂—C₁₋₃₀-alkyl, —NH₂, —NHR⁶, —NR⁶R⁷, —[NR⁶R⁷R⁸]⁺, —NH—COR⁶, —COON, —COOR⁶, —CONH₂, —CONHR⁶, —CONR⁶R⁷, —CO—H, —COR⁶, C₃₋₁₀-cycloalkyl, and C₆₋₁₄-aryl,
Rⁱⁱ at each occurrence are independently from each other selected from the group consisting of halogen, —CN, —NO₂, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR⁶, —S—C₁₋₃₀-alkyl, —NH₂, —NHR⁶, —NR⁶R⁷, —[NR⁶R⁷R⁸]⁺, —NH—COR⁶, —COOH, —COOR⁶, —CONH₂, —CONHR⁶, —CONR⁶R⁷, —CO—H, —COR⁶, C₁₋₃₀-alkyl, C₂₋₃₀-alkenyl, C₃₋₁₀-cycloalkyl, and C₆₋₁₄-aryl,
Rⁱⁱⁱ at each occurrence are independently from each other selected from the group consisting of halogen, —CN, —NO₂, —N₃, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR⁶, —S—C₁₋₃₀-alkyl, —SO₂—C₁₋₃₀-alkyl, —NH₂, —NHR⁶, —NR⁶R⁷, —[NR⁶R⁷R⁸]⁺, —NH—COR⁶, —COON, —COOR⁶, —CONH₂, —CONHR⁶, —CONR⁶R⁷, —CO—H, —COR⁶, C₁₋₃₀-alkyl, C₂₋₃₀-alkenyl, C₃₋₁₀-cycloalkyl, and C₆₋₁₄-aryl,
wherein
R⁶, R⁷ and R⁸ at each occurrence are independently from each other selected from the group consisting of C₁₋₃₀-alkyl, C₂₋₃₀-alkenyl, C₃₋₁₀-cycloalkyl, and C₆₋₁₄-aryl, and
X is as defined in claim 1.

3. The compound of claim 1, wherein
R¹ and R² are independently from each other C₁₋₃₀-alkyl optionally substituted with 1 to 30 substituents R^{a},
wherein
R^{a} at each occurrence are independently from each other selected from the group consisting of halogen, —CN, —NO₂, —N₃, —OH, C₁₋₃₀-alkoxy optionally substituted with 1 to 6 substituents Rⁱ, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl optionally substituted with 1 to 30 substituents Rⁱ, —SO₂—C₁₋₃₀-alkyl optionally substituted with 1 to 30 substituents Rⁱ, —NH₂, —NHR³, —NR³R⁴, —[NR³R⁴R⁵]⁺, —NH—COR³, —COOH, —COOR³, —CONH₂, —CONHR³, —CONR³R⁴, —CO—H, —COR³, C₃₋₁₀-cycloalkyl optionally substituted with 1 to 10 substituents Rⁱⁱ, and C₆₋₁₄-aryl optionally substituted with 1 to 8 substituents
wherein
R³, R⁴ and R⁵ at each occurrence are independently from each other selected from the group consisting of C₁₋₃₀-alkyl optionally substituted with 1 to 30 substituents Rⁱ, C₂₋₃₀-alkenyl optionally substituted with 1 to 30 substituents Rⁱ, C₃₋₁₀-cycloalkyl optionally substituted with 1 to 10 substituents Rⁱⁱ, and C₆₋₁₄-aryl optionally substituted with 1 to 8 substituents Rⁱⁱⁱ,
Rⁱ at each occurrence are independently from each other selected from the group consisting of halogen, —CN, —NO₂, —N₃, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR³, —S—C₁₋₃₀-alkyl, —SO₂—C₁₋₃₀-alkyl, —NH₂, —NHR⁶, —NR⁶R⁷, —[NR⁶R⁷R⁸]⁺, —NH—COR⁶, —COOH, —COOR⁶, —CONH₂, —CONHR⁶, —CONR⁶R⁷, —CO—H, —COR⁶, C₃₋₁₀-cycloalkyl, and C₆₋₁₄-aryl,
Rⁱⁱ at each occurrence are independently from each other selected from the group consisting of halogen, —CN, —NO₂, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR⁶, —S—C₁₋₃₀-alkyl, —NH₂, —NHR⁶, —NR⁶R⁷, —[NR⁶R⁷R⁸]⁺, —NH—COR⁶, —COOH, —COOR⁶, —CONH₂, —CONHR⁶, —CONR⁶R⁷, —CO—H, —COR⁶, C₁₋₃₀-alkyl, C₂₋₃₀-alkenyl, C₃₋₁₀-cycloalkyl, and C₆₋₁₄-aryl,
Rⁱⁱⁱ at each occurrence are independently from each other selected from the group consisting of halogen, —CN, —NO₂, —N₃, —OH, C₁₋₃₀-alkoxy, —O—[CH₂CH₂O]ₙ—C₁₋₁₀-alkyl (n=1 to 10), —O—[CH₂CH₂O]ₘ—OH (m=1 to 10), —O—COR⁶, —S—C₁₋₃₀-alkyl, —SO₂—C₁₋₃₀-alkyl, —NH₂, —NHR⁶, —NR⁶R⁷, —[NR⁶R⁷R⁸]⁺, —NH—COR⁶, —COOH, —COOR⁶, —CONH₂, —CONHR⁶, —CONR⁶R⁷, —CO—H, —COR⁶, C₁₋₃₀-alkyl, C₂₋₃₀-alkenyl, C₃₋₁₀-cycloalkyl, and C₆₋₁₄-aryl,
wherein
R⁶, R⁷ and R⁸ at each occurrence are independently from each other selected from the group consisting of C₁₋₃₀-alkyl, C₂₋₃₀-alkenyl, C₃₋₁₀-cycloalkyl, and C₆₋₁₄-aryl, and
X is as defined in claim 1.

4. The compound of claim 1, wherein
R¹ and R² are independently from each other C₃₋₂₅-alkyl branched at the C attached to the N of formula 1, and
X is as defined in claim 1.

5. A process for the preparation of a compound of formula 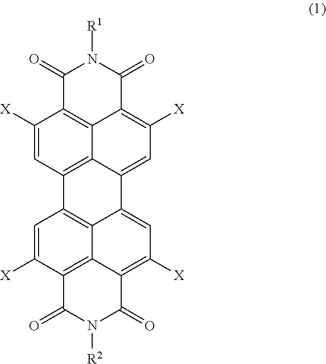
wherein R¹, R² and X are as defined in claim 1, which process comprises the steps of
(i) treating a compound of formula (2) with a boron-containing compound of formula (3) in the presence of a transition metal-containing catalyst to form a boron-containing compound of formula (4) 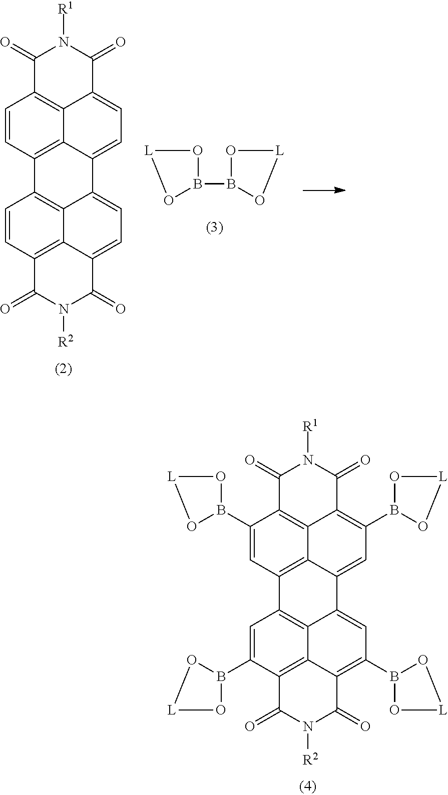
wherein R¹ and R² are as defined in claim 1, and L is a linking group,
and
(ii) treating the boron-containing compound of formula (4) with a Br-, Cl- or I-source in order to form the compound of formula (1).

6. An electronic device comprising the compound of formula (1) of any of claims 1 to 4 as semiconducting material.

7. Use of the compound of formula (1) of any of claims 1 to 4 as semiconducting material.
